(19) United States
(12) Patent Application Publication
(10) Pub. No.: US 2020/0306296 A1
(43) Pub. Date: Oct. 1, 2020

(54) INDUCED TISSUE REGENERATION USING EXTRACELLULAR VESICLES

(71) Applicant: AgeX Therapeutics, Inc., Alameda, CA (US)

(72) Inventors: Michael West, Mill Valley, CA (US); Dana Larocca, Alameda, CA (US); Jieun Lee, San Mateo, CA (US)

(73) Assignee: AgeX Therapeutics, Inc., Alameda, CA (US)

(21) Appl. No.: 16/833,285

(22) Filed: Mar. 27, 2020

Related U.S. Application Data
(60) Provisional application No. 62/825,732, filed on Mar. 28, 2019.
(60) Provisional application No. 62/872,246, filed on Jul. 9, 2019.

(51) Int. Cl. A61K 35/12

(52) U.S. Cl. CPC A61K 35/12

(57) ABSTRACT

Exosomes are generated from host embryonal carcinoma or embryonic progenitor cell lines and used for induced tissue regeneration. These inventive compositions and methods provide for induced tissue regeneration in aged or diseased mammalian tissues in vivo without the use of transgenically-expressed genes. Cells are reprogrammed to a prenatal, indeed a pre-fetal state, as indicated by for example in the case of many stromal cell types, by a decrease in the markers ADIRF and COX7A1.

### CROSS REFERENCE TO RELATED APPLICATIONS

This application claims priority benefit of the filing dates of U.S. Provisional Patent Application 62/825,732, filed Mar. 28, 2019, and U.S. Provisional Patent Application 62/872,246, filed Jul. 9, 2019, the contents of both of which are incorporated herein by reference in their entirely.

### FIELD OF THE INVENTION

The present invention relates to compositions and methods for the in vivo delivery of factors that modulate the developmental staging of cells and tissues in vivo. More specifically, the formulations utilizing human cell-derived extracellular vesicles described herein are designed to deliver a molecular cargo that alters the pattern of gene expression associated with the embryonic-fetal and neonatal transitions for therapeutic effect. The therapeutic outcome is an increase in the regenerative potential of mammalian tissues, an increase in the capacity for mammalian cells to undergo senolysis in vivo in some applications, and the modulation of the embryonic-fetal and neonatal transitions in tumors for improved therapeutic outcome of cancer in other applications.

### BACKGROUND

Advances in stem cell technology, such as the isolation and propagation in vitro of human pluripotent stem (hPS) cells, including but not limited to human embryonic stem (hES) and human induced pluripotent stem (hiPS) cells, constitute an important new area of medical research. hPS cells have a demonstrated potential to be propagated in the undifferentiated state or alternatively to be induced to differentiate into any and all of the cell types in the human body (Thomson et al., Science 282:1145-1147 (1998)). The unique intrinsic capacity of hPS cells to differentiate into all somatic cell types logically provides a platform for the manufacture of transplantable hPS-derived cells of similar diversity for the treatment for a wide variety of degenerative diseases. While this pluripotency of hES and hiPS cells is currently widely recognized, less recognized, and rarely studied, is the unique capacity of hPS cells cultured in vitro to generate relatively undifferentiated embryonic anlagen which, in the case of the human species, has historically been under-studied.

Even more rarely studied is the potential of hPS cell-derived cells to differentiate into what are commonly recognized as differentiated cell types such as cardiomyocytes or other diverse stromal cell types such as osteochondral cells, brown adipocytes, vascular cells and other cells that unexpectedly display subtle, largely unrecognized prenatal, or even pre-fetal patterns of gene expression that distinguish them from fetal or adult counterparts respectively.

Immediately prior to the embryonic-fetal transition (EFT) in vivo, mammalian differentiated cells and tissues such as the skin, heart, and spinal cord show a profound scarless regenerative potential that is progressively lost subsequent to the EFT. In the case of some tissues, such as the human heart, potential for scarless regeneration is detectable for approximately a week past the neonatal transition (NT) period. Given the importance of understanding and modulating tissue regeneration and tissue growth for the fields of regenerative medicine and oncology, improved methods for modelling and modulating the biology in vitro and in vivo have significant potential utility in research and clinical practice.

Epimorphic regeneration, sometimes referred to as “epimorphosis,” refers to a type of tissue regeneration wherein a blastema of relatively undifferentiated mesenchyme proliferates at the site of injury and then the cells differentiate to restore the original tissue histology. The developmental timing of the loss of epimorphic potential cannot be fixed precisely, and likely varies with tissue type, nevertheless, the EFT which occurs at about the end of eight weeks of human development (Carnegie Stage 23; O'Rahilly, R., F. Müller (1987) Developmental Stages in Human Embryos, Including a Revision of Streeter's ‘Horizons’ and a Survey of the Carnegie Collection. Washington, Carnegie Institution of Washington) appears to temporally correspond to the loss of skin regeneration in placental mammals (Walmsley, G. G. et al 2015. Scarless Wound Healing: Chasing the Holy Grail Plast Reconstr Surg. 135(3):907-17). Correlations between species show increased regenerative potential in the embryonic or larval state (reviewed in Morgan, T. H. (1901). Regeneration (New York: The MacMillan Company); also Sanchez Alvarado, A., and Tsonis, P. A. (2006) Bridging the regeneration gap: genetic insights from diverse animal models. Nat. Rev. Genet. 7, 873-884). This suggests that tissue regeneration, as opposed to scarring, reflects the presence of an embryonic as opposed to fetal or adult phenotype, though there is currently no consensus in the scientific community that epimorphic tissue regeneration is a result of an embryonic (pre-natal, more specifically, pre-fetal) pattern of gene expression. In the case of some species, a change in developmental timing (heterochrony) correlates with profound regenerative potential such as is the case in the developmental arrest in larval development (heterochrony) and limb regeneration observed in the Mexican salamander axolotl (A. mexicanum) (Voss, S. R. et al, Thyroid hormone responsive QTL and the evolution of paedomorphic salamanders. Heredity (2012) 109, 293-298.

We previously disclosed compositions and methods for reprogramming differentiated mammalian somatic cells to pluripotency, said methods including those related to the exposure of mammalian cells to cytoplasmic factors capable of reprogramming said somatic cells to pluripotency including the use of cytoplasmic blebs from undifferentiated cells expressing high levels of reprogramming factors such as BARX1, CROC4, DNMT3B, H2AFX, HHEX, HISTIH2AB, HISTH4J, HMGB2, hsa-miR-18a, hsa-miR-18b, hsa-miR-20b, hsa-miR-96, hsa-miR-106a, hsa-miR-107, hsa-miR-141, hsa-miR-183, hsa-miR-187, hsa-miR-203, hsa-miR-211, hsa-miR-217, hsa-miR-218-1, hsa-miR-218-2, hsa-miR-302a, hsa-miR-302c, hsa-miR-302d, hsa-miR-330, hsa-miR-363, hsa-miR-367, hsa-miR-371, hsa-miR-372, hsa-miR-373, hsa-miR-496, hsa-miR-50B, hsa-miR-512-3p, hsa-miR-512-5p, hsa-miR-515-3p, hsa-miR-515-5p, hsa-miR-516-5p, hsa-miR-517, hsa-miR-517a, 35 hsa-miR-518b, hsa-miR-51 Bc, hsa-miR-518e, hsa-miR-51 ge, hsa-miR-520a, hsa-miR-520b, hsa-miR-520e, hsa-miR-520g, hsa-miR-520h. hsa-miR-523, hsa-miR-524, hsa-miR-525, hsa-miR-526a-1, hsa-miR-526a-2, LEFTB, LHX 1, LHX6, LIN28, MYBL2, MYC, MYCN, NANOG, NFIX, OCT3/4 (POU5F1), OCT6 (POU3F1), OTX2, PHC1, SALL4, SOX2, TERFI, TERT, TGIF, VENTX2, ZIC2, ZIC3, ZIC5, and ZNF206 that transport said reprogramming factors (Methods of Reprogramming Animal Somatic Cells, U.S. Patent Application Publication 2017/0152475, incorporated in its entirety herein by reference).

In addition, we disclosed markers of the EFT in mammalian species and their use in partially reprogramming mammalian adult or fetal somatic cell types to a prenatal or even pre-fetal pattern of gene expression but without reprogramming the cells to pluripotency to modulate tissue regeneration and cancer progression described in International Patent Application Publication WO 2014/197421, titled “Compositions and Methods for Induced Tissue Regeneration in Mammalian Species,” which is incorporated herein by reference in its entirety and International Patent Application Publication WO 2017/214342, titled “Improved Methods for Detecting and Modulating the Embryonic-Fetal Transition in Mammalian Species,” which is incorporated herein by reference in its entirety. In addition, we disclosed small molecule modulators of the EFT and NT (International Patent Application PCT/US2019/028816, titled “Improved Methods for Inducing Tissue Regeneration and Senolysis in Mammalian Cells,” incorporated herein by reference in its entirety). The aforementioned compositions and methods were based in part on the methods allowing the clonal expansion of hPS cell-derived embryonic progenitor cell lines which provide a means to propagate novel diverse and highly purified cell lineages with a pre-natal and even pre-fetal patterns of gene expression useful for regenerating tissues such as skin in a scarless manner. Such cell types have important applications in research, and for the manufacture of cell-based therapies (see International Patent Application PCT/US2006/013519, filed on Apr. 11, 2006 and titled “Novel Uses of Cells With Prenatal Patterns of Gene Expression”; U.S. patent application Ser. No. 11/604,047, filed on Nov. 21, 2006 and titled “Methods to Accelerate the Isolation of Novel Cell Strains from Pluripotent Stem Cells and Cells Obtained Thereby”; and U.S. patent application Ser. No. 12/504,630 filed on Jul. 16, 2009 and titled “Methods to Accelerate the Isolation of Novel Cell Strains from Pluripotent Stem Cells and Cells Obtained Thereby”, each incorporated in its entirety herein by reference). Nevertheless, additional and improved methods and compositions for the in vivo delivery of said reprogramming factors useful in modulating the EFT and NT are needed. The present invention teaches compositions and methods related to the formulation, manufacture, storage, and delivery of human cell-derived extracellular vesicles delivering molecular cargo designed to modulate the EFT and NT for mammalian regenerative therapy and the treatment of diverse types of malignancy.

Extracellular vesicles (EVs), also known as extracellular blebs [U.S. Patent Application Publication 2017/0152475, titled “Methods of Reprogramming Animal Somatic Cells,” which is incorporated in its entirety herein by reference) are generally classified into three sub-categories: 1) Exosomes which range from 30-150 nM in diameter; 2) microvesicles or ectosomes which range from 50 nm-1.0 μM; and apoptotic blebs also known as apoptotic bodies which range from 50 nm-5.0 μM. Exosomes are distinguished from ectosomes by their biogenesis with exosomes being shed by exocytosis of multivesicular bodies and ectosomes through a budding process at the cell membrane (Trends Cell Biol 2015, 25(6):364-72). EVs are believed to contain important signaling molecules that may provide the source of trophic factors responsible for some regenerative benefits seen in cell replacement therapy. As such they would provide an alternative to some cell based therapies with lower costs for manufacture to scale, lower risks of immune rejection, and potentially affecting multiple cell types allowing for complex tissue regeneration involving a complex interaction of cells not easily introduced as a cell-based therapy. Accordingly, EVs may provide an attractive alternative or adjunct to cell based therapies and cell based regenerative medicine.

Exosomes are 30 to 150 nm EVs secreted by a wide range of mammalian cell types. Keller et al. (2006) Immunol Lett. 107(2):102; Camussi et al. (2010) Kidney International 78:838. The vesicles are enclosed by a lipid bilayer and are larger than LDL which has a size of 22 nm, but smaller than a red blood cell, which is 6000 to 8000 nm in diameter and has a thickness of 2000 nm. Keller et al. (2006) Immunol Lett. 107(2):102.

Exosomes are found both in cells growing in vitro as well as in vivo. They can be isolated from tissue culture media as well as bodily fluids such as plasma, urine, milk and cerebrospinal fluid. George et al. (1982) Blood 60:834; Martinez et al. (2005) Am J Physiol Health Cir Physiol 288:H1004. Exosomes originate from the endosomal membrane compartment. They are stored in intraluminal vesicles within multivesicular bodies of the late endosome. Multivesicular bodies are derived from the early endosome compartment and contain within them smaller vesicular bodies that include exosomes. Exosomes are released from the cell when multivesicular bodies fuse with the plasma membrane. Methods of isolating exosomes from cells have been described, see e.g., US Patent Application Publication 2012/0093885.

Exosomes contain a variety of molecules including proteins, lipids and nucleic acids such as DNA, mRNA and miRNA. Their contents are believed to play a part in cell to cell communication involving the release of the exosome from one cell and the binding/fusion of the exosome with a second cell, wherein the contents of the exosomal compartment are released within the second cell.

It has been reported that exosomes derived from human adult circulating endothelial progenitor cells may act as vehicle for mRNA transport among cells. These exosomes were shown to incorporate into normal endothelial cells by interacting with the α4β1 integrin. Once incorporated into the endothelial cells, the exosomes stimulated an angiogenic program. Deregibus et al. (2007) Blood 110:2440. Similar results were obtained in vivo using severe combined immunodeficient mice. Exosome stimulated endothelial cells implanted subcutaneously in Matrigel (a murine sarcoma extract) organized into a patent vessel network connected with the murine vasculature. Deregibus, supra.; Bruno et al. (2009) J Am Soc Nephrol 20:1053; Herrera et al. (2010) J Cell Mol Med 14:1605.

Of the various molecular cargo of exosomes, miRNAs have recently attracted a lot of attention due to their regulatory roles in gene expression. MiRNAs are small, non-coding regulatory RNAs that can have a wide range of effects on multiple RNA targets, thus having the potential to have greater phenotypic influence than coding RNAs. MiRNA profiles of exosomes often differ from those of the parent cells. Profiling studies have demonstrated that miRNAs are not randomly incorporated into exosomes but rather a subset of miRNAs is preferentially packaged into exosomes, suggesting an active sorting mechanism of exosomal miRNAs. Guduric-Fuchs et al. (2014) Nucleic Acid Res. 42:9195; Ohshima et al. (2010) PloS One 5(10):e13247.

Because exosomes contain a variety of molecules, many believed to play an important role in cell signaling, exosomes would prove useful in research and industry and would have applications as therapeutics, diagnostics and in screening assays. Frequently, however, the availability of reproducible, essentially identical populations of exosomes is limited by the fact that most sources of exosomes are cells that senesce and thus have limited replicative capacity. Accordingly, there is a need for exosomes that are derived from a clonal source that has an extended replicative capacity that is greater than most adult or fetal derived cells. The invention described infra meets this need and as well as other needs in the field.

Age reversal methods using partial reprogramming described in the literature by viral and other gene therapy vectors to force expression of reprogramming factors have risks of unwanted gene modifications and potential for unwanted cell growth such as tumor formation.

Exosomes are potentially safer and more cost effective to produce. There are approaches to exosome treatments that do not use clonal progenitor cell lines or consider modulating EFT markers.

There is a need in the art for improved mechanisms to modulate global alterations in gene expression associated with the EFT and NT in order to induce cell and tissue regeneration and reprogramming and to alter the course of cancer. There is therefore a further need for improved methods of formulating EFT and NT developmental reprogramming factors that is both cost-effective and capable of distributing the factors systemically in vivo.

### SUMMARY OF THE INVENTION

One aspect of the present invention is compositions and methods wherein one or more extracellular vesicles are utilized in the induction of tissue regeneration in mammals in vivo.

In one preferred embodiment, the extracellular vesicles in the compositions and methods of the invention are generated by embryonic progenitor cell lines. Said embryonic progenitor cell lines may in one embodiment be human pluripotent stem cell-derived clonal embryonic progenitor cell lines that lack expression of the fetal-adult marker COX7A1, lack expression of the NT marker ADIRF, and express the embryonic marker PCDHB2.

Another aspect of the invention is regenerated cells or tissue induced by extracellular vesicles without the use of transgenic gene-based forced expression of pluripotency factors.

Another aspect of the invention is regenerated cells or tissue induced by isolated extracellular vesicles that have been loaded with reprogramming factors after the isolation of the extracellular vesicles.

One aspect of the present invention is one or more exosomes utilized in the induction of tissue regeneration.

In one preferred embodiment, the exosomes in the compositions and methods of the invention are generated by embryonic progenitor cell lines. Said embryonic progenitor cell lines may in one embodiment be human pluripotent stem cell-derived clonal embryonic progenitor cell lines that lack expression of the fetal-adult marker COX7A1, lack expression of the NT marker ADIRF, and express the embryonic marker PCDHB2.

Another aspect of the invention is regenerated cells or tissue induced by exosomes without the use of transgenic gene-based forced expression of pluripotency factors.

Another aspect of the invention is regenerated cells or tissue induced by isolated exosomes that have been loaded with reprogramming factors after the isolation of the exosomes.

In one preferred embodiment, the extracellular vesicles in the compositions and methods of the invention are generated by embryonal carcinoma cell lines. Said embryonal carcinoma cell lines may in one embodiment be embryonal carcinoma cell lines that lack expression of the fetal-adult marker COX7A1, lack expression of the NT marker ADIRF, and express pluripotency markers such as OCT4 and TERT.

Another aspect of the invention is regenerated cells or tissue induced by extracellular vesicles derived from embryonal carcinoma cell lines without the use of transgenic gene-based forced expression of reprogramming factors.

Another aspect of the invention is regenerated cells or tissue induced by isolated extracellular vesicles derived from embryonal carcinoma cell lines that have been loaded with reprogramming factors chosen from the list BARX1, CROC4, DNMT3B, H2AFX, HHEX, HISTIH2AB, HISTIH4J, HMGB2, hsa-miR-18a, hsa-miR-18b, hsa-miR-20b, hsa-miR-96, hsa-miR-106a, hsa-miR-107, hsa-miR-141, hsa-miR-183, hsa-miR-187, hsa-miR-200, hsa-miR-203, hsa-miR-211, hsa-miR-217, hsa-miR-218-1, hsa-miR-218-2, hsa-miR-290, hsa-miR-294, hsa-miR-295. hsa-miR-302a, hsa-miR-302b, hsa-miR-302c, hsa-miR-302d, hsa-miR-330, hsa-miR-363, hsa-miR-367, hsa-miR-371, hsa-miR-372, hsa-miR-373, hsa-miR-496, hsa-miR-50B, hsa-miR-512-3p, hsa-miR-512-5p, hsa-miR-515-3p, hsa-miR-515-5p, hsa-miR-516-5p, hsa-miR-517, hsa-miR-517a, 35 hsa-miR-518b, hsa-miR-51Bc, hsa-miR-518e, hsa-miR-51 ge, hsa-miR-520a, hsa-miR-520b, hsa-miR-520e, hsa-miR-520g, hsa-miR-520h, hsa-miR-523, hsa-miR-524, hsa-miR-525, hsa-miR-526a-1, hsa-miR-526a-2, LEFTB, LHX1, LHX6, LIN28, MYBL2, MYC, MYCN, NANOG, NFIX, OCT3/4 (POU5F1), OCT6 (POU3F1), OTX2, PHC1, SALL4, SOX2, TERF 1, TERT, TGIF, VENTX2, ZIC2, ZIC3, ZIC5, and ZNF206 after the isolation of the extracellular vesicles.

One aspect of the present invention is one or more exosomes derived from embryonal carcinoma cell lines utilized in the induction of tissue regeneration.

Another aspect of the invention are cells reprogrammed in vivo to an embryonic/pre-fetal-like state (pre-EFT (embryonic to fetal transition)) but are not reprogrammed to pluripotency (now described in the literature as “partial reprogramming.” In a preferred embodiment, the reprogramming or pre-EFT state is indicated by a decrease in the EFT marker COX7A1 and the NT marker ADIRF (1).

In a method according to the invention, extracellular vesicles such as exosomes, from embryonic progenitor cells or pluripotent cells such as hES cells or hEC cells that are screened for ability to confer pre-EFT gene expression pattern, are contacted with fetal/adult cells and/or tissue to restore regenerative capacity in a subject in vivo. Said subject may be human or non-human mammal. In one embodiment the subject is an aging or elderly human. In a further embodiment, the method prevents, slows or reverses the loss of regenerative capacity in aging adult cells and/or tissue. In a another preferred embodiment, the method according to the invention can partially or fully reset or improve various hallmarks of aging such as epigenetic profile, telomere length and/or mitochondrial fitness to an embryonic state.

### BRIEF DESCRIPTION OF THE DRAWINGS

For a fuller understanding of the nature and advantages of the present invention, reference should be had to the following detailed description taken in connection with the accompanying drawings, in which:

FIG. 1 is a chart showing the EFT marker COX7A1 is down regulated early in the process of OKSM reprogramming. The gene expression data for COX7A1 (from Ohnuki et al. (9)) was plotted onto a graph of eAge change with reprogramming adapted from FIG. 1a of Olova et al (10). COX7A1 expression (solid black line) reaches ˜10% of maximum when the cells are partially reprogrammed and at day 20 when cells reach epigenetic age of 0 (black heavy dashed line) and are incompletely reprogrammed. Expression of COX7A1 is minimal at days 28-49 (fully reprogrammed). The newborn/adult iTR marker ADIRF also rapidly decreases in expression early in reprogramming. In contrast, the embryonic stem cell marker, TERT, increases in expression starting at day 20 when cells are reset to epigenetic age near zero and continues to be expressed in cells reprogrammed to pluripotency.

FIGS. 2A and 2B are charts showing that conditioned medium from endothelial embryonic progenitor cells confers an embryonic phenotype onto fetal/adult endothelial cells as indicated by EFT marker COX7A1. Decrease in COX7A1 expression following 72-hour incubation of HUVEC cells (FIG. 2A) or HBMVEC cells (FIG. 2B) with conditioned medium from 30-MV2-10, 30-MV2-17, and 30-MV2-19 eEPC.

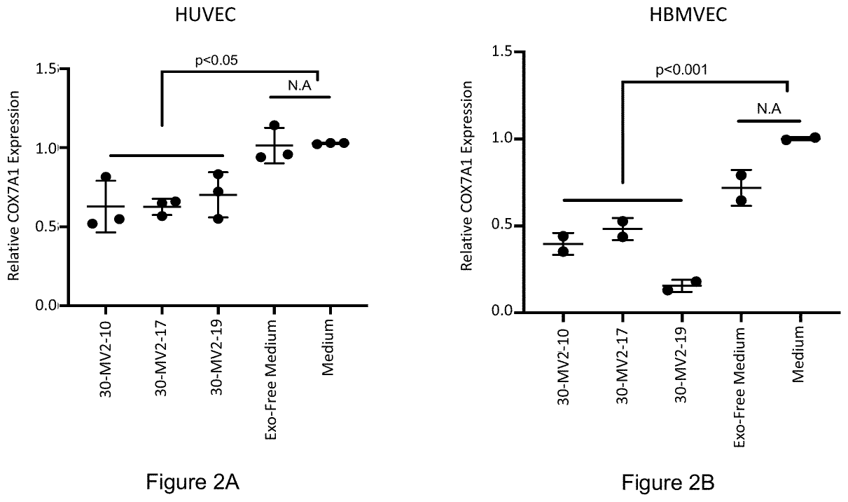

FIGS. 3A and 3B are charts showing that exosome preparations from endothelial embryonic progenitor cells confer an embryonic phenotype onto fetal/adult endothelial cells as indicated by EFT Marker COX7A1. Decrease in COX7A1 expression following 6-day treatment of (FIG. 3A) HUVEC or (FIG. 3B) HBMEC (Human Brain Microvascular Endothelial Cells) with exosome enriched conditioned medium from embryonic endothelial progenitor cells (30-MV2-10, 30-MV2-14, and 30-MV2-19). No significant effect is seen when HUVEC or HBMEC cells are treated with their own exosome enriched conditioned medium from HUVEC or HBMEC respectively.

### DETAILED DESCRIPTION

### Abbreviations

AC—Adult-derived cells

AMH—Anti-Mullerian Hormone

ASC—Adult stem cells

cGMP—Current Good Manufacturing Processes

CM—Cancer Maturation

CNS—Central Nervous System

DMEM—Dulbecco's modified Eagle's medium

DMSO—Dimethyl sulphoxide

DNAm—Changes in the methylation of DNA that provide a marker or “clock” of the age of cells and tissue.

DPBS—Dulbecco's Phosphate Buffered Saline

ED Cells—Embryo-derived cells; hED cells are human ED cells

EDTA—Ethylenediamine tetraacetic acid

EFT—Embryonic-Fetal Transition

EG Cells—Embryonic germ cells; hEG cells are human EG cells

EP—Embryonic progenitors

ES Cells—Embryonic stem cells; hES cells are human ES cells

ESC—Embryonic Stem Cells

EVs—Extracellular Vesicles

FACS—Fluorescence activated cell sorting

FBS—Fetal bovine serum

FPKM—Fragments Per Kilobase of transcript per Million mapped reads from RNA sequencing.

GFP—Green fluorescent protein

GMP—Good Manufacturing Practices

HAEC—Human Aortic Endothelial Cell

hEC Cells—Human Embryonal Carcinoma Cells

hED Cells—Human embryo-derived cells

hEG Cells—“Human embryonic germ cells” are stem cells derived from the primordial germ cells of fetal tissue.

HESC—Human Embryonic Stem Cells including human ES-like cells, therefore both primed and naïve pluripotent stem cells.

hiPS Cells—“Human induced pluripotent stem cells” are cells with properties similar to hES cells obtained from somatic cells after exposure to hES-specific transcription factors such as SOX2, KLF4, OCT4, MYC, or NANOG, LIN28, OCT4, and SOX2.

HPS Cells—Human pluripotent stem cells such as hES cells, hiPS cells, EC cells, and human parthenogenic stem cells.

HSE—“Human skin equivalents” are mixtures of cells and biological or synthetic matrices manufactured for testing purposes or for therapeutic application in promoting wound repair.

iCM—Induced Cancer Maturation.

iPS Cells—“Induced pluripotent stem cells” are cells with properties similar to hES cells obtained from somatic cells after exposure to ES-specific transcription factors such as SOX2. KLF4, OCT4, MYC, or NANOG, LIN28, OCT4, and SOX2. SOX2, KLF4, OCT4, MYC, and (LIN28A or LIN28B), or other combinations of OCT4, SOX2, KLF4, NANOG, ESRRB, NRSA2, CEBPA, MYC, LIN28A and LIN28B.

iS-CSC—“Induced Senolysis of Cancer Stem Cells” refers to the treatment of cells in malignant tumors that are refractory to ablation by chemotherapeutic agents or radiation therapy wherein said iS-CSC treatment causes said refractory cells to revert to a pre-fetal pattern of gene expression and become sensitive to chemotherapeutic agents or radiation therapy.

iTM—Induced Tissue Maturation

iTR—Induced Tissue Regeneration

MEM—Minimal essential medium

MSC—Mesenchymal stem cell

NT—Neonatal Transition

PBS—Phosphate buffered saline

PS fibroblasts—“Pre-scarring fibroblasts” are fibroblasts derived from the skin of early gestational skin or derived from ED cells that display a prenatal pattern of gene expression in that they promote the rapid healing of dermal wounds without scar formation.

RFU—Relative Fluorescence Units

RNA-seq—RNA sequencing

SFM—Serum-Free Medium

St. Dev.—Standard Deviation

TR—Tissue Regeneration

### Definitions

The term “analytical reprogramming technology” refers to a variety of methods to reprogram the pattern of gene expression of a somatic cell to that of a more pluripotent state, such as that of an iPS, ES, ED, EC or EG cell, wherein the reprogramming occurs in multiple and discrete steps and does not rely simply on the transfer of a somatic cell into an oocyte and the activation of that oocyte (see U.S. application Nos. 60/332,510, filed Nov. 26, 2001; Ser. No. 10/304,020, filed Nov. 26, 2002; PCT application no. PCT/US02/37899, filed Nov. 26, 2003; U.S. application No. 60/705,625, filed Aug. 3, 2005; U.S. application No. 60/729,173, filed Aug. 20, 2005; U.S. application No. 60/818,813, filed Jul. 5, 2006, PCT/US06/30632, filed Aug. 3, 2006, the disclosure of each of which is incorporated by reference herein).

The term “blastomere/morula cells” refers to blastomere or morula cells in a mammalian embryo or blastomere or morula cells cultured in vitro with or without additional cells including differentiated derivatives of those cells.

The term “induced Senolysis of Cancer Stem Cells” (iS-CSC) refers to the treatment of cells in malignant tumors that are refractory to ablation by chemotherapeutic agents or radiation therapy wherein said iS-CSC treatment causes said refractory cells to revert to a pre-fetal pattern of gene expression and become sensitive to chemotherapeutic agents or radiation therapy.

The term “cell expressing gene X”, “gene X is expressed in a cell” (or cell population), or equivalents thereof, means that analysis of the cell using a specific assay platform provided a positive result. The converse is also true (i.e., by a cell not expressing gene X, or equivalents, is meant that analysis of the cell using a specific assay platform provided a negative result). Thus, any gene expression result described herein is tied to the specific probe or probes employed in the assay platform (or platforms) for the gene indicated.

The term “exosome” refers to an extracellular vesicle with a size range of 30-100 nm that is secreted by cells and transports a mixture of molecular components as cargo, said mixture comprising a combination of RNAs, including miRNAs, lipids and proteins.

The term “ectosome” or “microsome” refers to an extracellular vesicle with a size range of 50 nm-1.0 μm that is secreted by cells and transports a mixture of molecular components as cargo, said mixture comprising a combination of RNAs, including miRNAs, lipids and proteins.

The term “apoptotic body” refers to an extracellular vesicle with a size range of 50 nm-5.0 μm that results from the apoptosis of cells and transports a mixture of molecular components as cargo, said mixture comprising a combination of RNAs, including miRNAs, and proteins.

The term “cytoplasmic bleb” or “extracellular vesicle” is a general term that refers to the cytoplasm of a cell bound by an intact, or permeabilized, but otherwise intact plasma membrane but lacking a nucleus. Subtypes of vesicles all of which are included in the terms “cytoplasmic bleb” or “extracellular vesicle” include the exosome, ectosome/microsome, and apoptotic body. The terms are also used interchangeably and synonymously with the term “enucleate cytoplast” and “enucleate(d) cytoplasm”, unless the term “enucleate cytoplasm” is explicitly used in the context of an extract in which the plasma membrane has been removed.

The term “cell line” refers to a mortal or immortal population of cells that is capable of propagation and expansion in vitro.

The term “clonal” refers to a population of cells obtained by the expansion of a single cell into a population of cells all derived from that original single cells and not containing other cells.

The term “differentiated cells” when used in reference to cells made by methods of this invention from pluripotent stem cells refer to cells having reduced potential to differentiate when compared to the parent pluripotent stem cells. The differentiated cells of this invention comprise cells that could differentiate further (i.e., they may not be terminally differentiated).

The term “embryonic” or “embryonic stages of development” refers to prenatal stages of development of cells, tissues or animals, specifically, the embryonic phases of development of cells compared to fetal and adult cells. In the case of the human species, the transition from embryonic to fetal development occurs at about 8 weeks of prenatal development, in mouse it occurs on or about 16 days, and in the rat species, at approximately 17.5 days post coitum. (http://php.med.unsw.edu.au/embryology/index.php?title=Mouse_Timeline_Detailed).

The term “embryonic stem cells” (ES cells) refers to cells derived from the inner cell mass of blastocysts, blastomeres, or morulae that have been serially passaged as cell lines while maintaining an undifferentiated state (e.g. expressing TERT. OCT4, and SSEA and TRA antigens specific for ES cells of the species). The ES cells may be derived from fertilization of an egg cell with sperm or DNA, nuclear transfer, parthenogenesis, or by means to generate hES cells with hemizygosity or homozygosity in the MHC region. While ES cells have historically been defined as cells capable of differentiating into all of the somatic cell types as well as germ line when transplanted into a preimplantation embryo, candidate ES cultures from many species, including human, have a more flattened appearance in culture and typically do not contribute to germ line differentiation, and are therefore called “ES-like cells.” It is commonly believed that human ES cells are in reality “ES-like”, however, in this application we will use the term ES cells to refer to both ES and ES-like cell lines.

The term “human parthenogenetic stem cells” refers to pluripotent stem cells derived from an unfertilized but activated oocyte.

The term “global modulator of TR” or “global modulator of iTR” refers to agents capable of modulating a multiplicity of iTR genes or iTM genes including, but not limited to, agents capable of downregulating COA7A1 while simultaneously up-regulating PCDHB2, or down-regulating NAALADL1 while simultaneously up-regulating AMH in cells derived from fetal or adult sources and are capable of inducing a pattern of gene expression leading to increased scarless tissue regeneration in response to tissue damage or degenerative disease.

The term “human embryo-derived” (“hED”) cells refers to blastomere-derived cells, morula-derived cells, blastocyst-derived cells including those of the inner cell mass, embryonic shield, or epiblast, or other totipotent or pluripotent stem cells of the early embryo, including primitive endoderm, ectoderm, mesoderm, and neural crest and their derivatives up to a state of differentiation correlating to the equivalent of the first eight weeks of normal human development, but excluding cells derived from hES cells that have been passaged as cell lines (see, e.g., U.S. Pat. Nos. 7,582,479; 7,217,569; 6,887,706; 6,602,711; 6,280,718; and U.S. Pat. No. 5,843,780 to Thomson). The hED cells may be derived from preimplantation embryos produced by fertilization of an egg cell with sperm or DNA, nuclear transfer, or chromatin transfer, an egg cell induced to form a parthenote through parthenogenesis, analytical reprogramming technology, or by means to generate hES cells with hemizygosity or homozygosity in the HLA region. The term “human embryonic germ cells” (hEG cells) refer to pluripotent stem cells derived from the primordial germ cells of fetal tissue or maturing or mature germ cells such as oocytes and spermatogonial cells, that can differentiate into various tissues in the body. The hEG cells may also be derived from pluripotent stem cells produced by gynogenetic or androgenetic means, i.e., methods wherein the pluripotent cells are derived from oocytes containing only DNA of male or female origin and therefore will comprise all female-derived or male-derived DNA.

The term “human embryonic stem cells” (hES cells) refers to human ES cells.

The term “human embryonal carcinoma cells” (hEC cells) refers to cell lines derived from embryonal carcinomas including teratocarcinomas that express pluripotency markers but have chromosomal aberrations and are malignant in nature.

The term “human induced pluripotent stem cells” refers to cells with properties similar to hES cells, including the ability to form all three germ layers when transplanted into immunocompromised mice wherein said iPS cells are derived from cells of varied somatic cell lineages following exposure to de-differentiation factors, for example hES cell-specific transcription factor combinations: KLF4, SOX2, MYC; OCT4 or SOX2, OCT4, NANOG, and LIN28; or various combinations of OCT4, SOX2. KLF4, NANOG, ESRRB, NR5A2, CEBPA, MYC, LIN28A and LIN28B or other methods that induce somatic cells to attain a pluripotent stem cell state with properties similar to hES cells. However, the reprogramming of somatic cells by somatic cell nuclear transfer (SCNT) are typically referred to as NT-ES cells as opposed to iPS cells.

The term “induced Cancer Maturation” refers to methods resulting in a change in the phenotype of premalignant or malignant cells such that subsequent to said induction, the cells express markers normally expressed in that cell type in fetal or adult stages of development as opposed to the embryonic stages.

The term “induced tissue regeneration” refers to the use of the methods of the present invention to alter the molecular composition of fetal or adult mammalian cells such that said cells are capable or regenerating functional tissue following damage to that tissue wherein said regeneration would not be the normal outcome in animals of that species. While functionally iTR is intended to generate new tissue formation at the sights of injury or degenerative disease or to induce senolysis in CSCs or aged cells, the inventors of the present invention teach that in iTR is in fact reversing many aspects of aging in cells including markers such as DNAm but not restoring telomerase activity. The addition of telomerase activity together with iTR is also defined in the present invention as “iTR”.

The term “iTR-related Senolysis” refers to the induction of apoptosis in cells of aged tissues that have significant DNA damage including but not limited to that from cell aging (telomere shortening) through the reprogramming of said damaged cells to an embryonic pattern of gene expression.

The term “isolated” refers to a substance that is (i) separated from at least some other substances with which it is normally found in nature, usually by a process involving the hand of man, (ii) artificially produced (e.g., chemically synthesized), and/or (iii) present in an artificial environment or context (i.e., an environment or context in which it is not normally found in nature).

The term “iCM factors” refers to molecules that alter the levels of CM activators and CM inhibitors in a manner leading to CM in a tumor for therapeutic effect.

The term “iCM genes” refers to genes that when altered in expression can cause CM in a tumor for therapeutic effect.

The term “iS-CSC factors” refers to molecules that alter the levels of TR activators and TR inhibitors in a manner leading to TR and associated increase in sensitivity to apoptosis of cancer cells exposed to chemotherapeutic or radiation therapy.

The term “iTR factor” refers to molecules that alter the levels of TR activators and TR inhibitors in a manner leading to TR in a tissue not naturally capable of TR. Said iTR factor also refers to combinations of individual factors. Therefore, cocktails of factors described herein are considered an “iTR factor” in the present application.

The term “iTR genes” refers to genes that when altered in expression can cause induced tissue regeneration in tissues not normally capable of such regeneration.

The term “nucleic acid” is used interchangeably with “polynucleotide” and encompasses in various embodiments naturally occurring polymers of nucleosides, such as DNA and RNA, and non-naturally occurring polymers of nucleosides or nucleoside analogs. In some embodiments a nucleic acid comprises standard nucleosides (abbreviated A, G, C, T, U). In other embodiments, a nucleic acid comprises one or more non-standard nucleosides. In some embodiments, one or more nucleosides are non-naturally occurring nucleosides or nucleotide analogs. A nucleic acid can comprise modified bases (for example, methylated bases), modified sugars (2′-fluororibose, arabinose, or hexose), modified phosphate groups or other linkages between nucleosides or nucleoside analogs (for example, phosphorothioates or 5′-N-phosphoramidite linkages), locked nucleic acids, or morpholinos. In some embodiments, a nucleic acid comprises nucleosides that are linked by phosphodiester bonds, as in DNA and RNA. In some embodiments, at least some nucleosides are linked by non-phosphodiester bond(s). A nucleic acid can be single-stranded, double-stranded, or partially double-stranded. An at least partially double-stranded nucleic acid can have one or more overhangs, e.g., 5′ and/or 3′ overhang(s). Nucleic acid modifications (e.g., nucleoside and/or backbone modifications, including use of non-standard nucleosides) known in the art as being useful in the context of RNA interference (RNAi), aptamer, or antisense-based molecules for research or therapeutic purposes are contemplated for use in various embodiments of the instant invention. See, e.g., Crooke, S T (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008. In some embodiments, a modification increases half-life and/or stability of a nucleic acid, e.g., in vivo, relative to RNA or DNA of the same length and strandedness. In some embodiments, a modification decreases immunogenicity of a nucleic acid relative to RNA or DNA of the same length and strandedness. In some embodiments, between 5% and 95% of the nucleosides in one or both strands of a nucleic acid is modified. Modifications may be located uniformly or nonuniformly, and the location of the modifications (e.g., near the middle, near or at the ends, alternating, etc.) can be selected to enhance desired property(ies). A nucleic acid may comprise a detectable label, e.g., a fluorescent dye, radioactive atom, etc. “Oligonucleotide” refers to a relatively short nucleic acid, e.g., typically between about 4 and about 60 nucleotides long. Where reference is made herein to a polynucleotide, it is understood that both DNA, RNA, and in each case both single- and double-stranded forms (and complements of each single-stranded molecule) are provided.

“Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The term “oligoclonal” refers to a population of cells that originated from a small population of cells, typically 2-1000 cells, that appear to share similar characteristics such as morphology or the presence or absence of markers of differentiation that differ from those of other cells in the same culture. Oligoclonal cells are isolated from cells that do not share these common characteristics, and are allowed to proliferate, generating a population of cells that are essentially entirely derived from the original population of similar cells.

The term “pluripotent stem cells” refers to animal cells capable of differentiating into more than one differentiated cell type. Such cells include hES cells, blastomere/morula cells and their derived hED cells, hiPS cells, hEG cells, hEC cells, and adult-derived cells including mesenchymal stem cells, neuronal stem cells, and bone marrow-derived stem cells. Pluripotent stem cells may be genetically modified or not genetically modified. Genetically modified cells may include markers such as fluorescent proteins to facilitate their identification within the egg.

The term “polypeptide” refers to a polymer of amino acids. The terms “protein” and “polypeptide” are used interchangeably herein. A peptide is a relatively short polypeptide, typically between about 2 and 60 amino acids in length. Polypeptides used herein typically contain the standard amino acids (i.e., the 20 L-amino acids that are most commonly found in proteins). However, a polypeptide can contain one or more non-standard amino acids (which may be naturally occurring or non-naturally occurring) and/or amino acid analogs known in the art in certain embodiments. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide”. Polypeptides may be purified from natural sources, produced using recombinant DNA technology, synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated. A polypeptide may be cyclic or contain a cyclic portion. Where a naturally occurring polypeptide is discussed herein, it will be understood that the invention encompasses embodiments that relate to any isoform thereof (e.g., different proteins arising from the same gene as a result of alternative splicing or editing of mRNA or as a result of different alleles of a gene, e.g., alleles differing by one or more single nucleotide polymorphisms (typically such alleles will be at least 95%, 96%, 97%, 98%, 99%, or more identical to a reference or consensus sequence). A polypeptide may comprise a sequence that targets it for secretion or to a particular intracellular compartment (e.g., the nucleus) and/or a sequence targets the polypeptide for post-translational modification or degradation. Certain polypeptides may be synthesized as a precursor that undergoes post-translational cleavage or other processing to become a mature polypeptide. In some instances, such cleavage may only occur upon particular activating events. Where relevant, the invention provides embodiments relating to precursor polypeptides and embodiments relating to mature versions of a polypeptide.

The term “pooled clonal” refers to a population of cells obtained by combining two or more clonal populations to generate a population of cells with a uniformity of markers such as markers of gene expression, similar to a clonal population, but not a population wherein all the cells were derived from the same original clone. Said pooled clonal lines may include cells of a single or mixed genotypes. Pooled clonal lines are especially useful in the cases where clonal lines differentiate relatively early or alter in an undesirable way early in their proliferative lifespan.

The term “prenatal” refers to a stage of embryonic development of a placental mammal prior to which an animal is not capable of viability apart from the uterus.

The term “primordial stem cells” refers collectively to pluripotent stem cells capable of differentiating into cells of all three primary germ layers: endoderm, mesoderm, and ectoderm, as well as neural crest. Therefore, examples of primordial stem cells would include but not be limited by human or non-human mammalian ES cells or cell lines, blastomere/morula cells and their derived ED cells, iPS, and EG cells.

The term “purified” refers to agents or entities (e.g., compounds) that have been separated from most of the components with which they are associated in nature or when originally generated. In general, such purification involves action of the hand of man. Purified agents or entities may be partially purified, substantially purified, or pure. Such agents or entities may be, for example, at least 50%, 60%, 70%, 75%, 80%, 85%, 90%, 95%, 96%, 97%, 98%, 99%, or more than 99% pure. In some embodiments, a nucleic acid or polypeptide is purified such that it constitutes at least 75%, 80%, 85%, 90%, 95%, 96%, 97%, 98%, 99%, or more, of the total nucleic acid or polypeptide material, respectively, present in a preparation. Purity can be based on, e.g., dry weight, size of peaks on a chromatography tracing, molecular abundance, intensity of bands on a gel, or intensity of any signal that correlates with molecular abundance, or any art-accepted quantification method. In some embodiments, water, buffers, ions, and/or small molecules (e.g., precursors such as nucleotides or amino acids), can optionally be present in a purified preparation. A purified molecule may be prepared by separating it from other substances (e.g., other cellular materials), or by producing it in such a manner to achieve a desired degree of purity. In some embodiments, a purified molecule or composition refers to a molecule or composition that is prepared using any art-accepted method of purification. In some embodiments “partially purified” means that a molecule produced by a cell is no longer present within the cell, e.g., the cell has been lysed and, optionally, at least some of the cellular material (e.g., cell wall, cell membrane(s), cell organelle(s)) has been removed.

The term “RNA interference” (RNAi) is used herein consistently with its meaning in the art to refer to a phenomenon whereby double-stranded RNA (dsRNA) triggers the sequence-specific degradation or translational repression of a corresponding mRNA having complementarity to a strand of the dsRNA. It will be appreciated that the complementarity between the strand of the dsRNA and the mRNA need not be 100% but need only be sufficient to mediate inhibition of gene expression (also referred to as “silencing” or “knockdown”). For example, the degree of complementarity is such that the strand can either (i) guide cleavage of the mRNA in the RNA-induced silencing complex (RISC); or (ii) cause translational repression of the mRNA. In certain embodiments the double-stranded portion of the RNA is less than about 30 nucleotides in length, e.g., between 17 and 29 nucleotides in length. In certain embodiments a first strand of the dsRNA is at least 80%, 85%, 90%, 95%, or 100% complementary to a target mRNA and the other strand of the dsRNA is at least 80%, 85%, 90%, 95%, or 100% complementary to the first strand. In mammalian cells, RNAi may be achieved by introducing an appropriate double-stranded nucleic acid into the cells or expressing a nucleic acid in cells that is then processed intracellularly to yield dsRNA therein. Nucleic acids capable of mediating RNAi are referred to herein as “RNAi agents”. Exemplary nucleic acids capable of mediating RNAi are a short hairpin RNA (shRNA), a short interfering RNA (siRNA), and a microRNA precursor. These terms are well known and are used herein consistently with their meaning in the art. siRNAs typically comprise two separate nucleic acid strands that are hybridized to each other to form a duplex. They can be synthesized in vitro, e.g., using standard nucleic acid synthesis techniques. siRNAs are typically double-stranded oligonucleotides having 16-30, e.g., 16, 17, 18, 19, 20, 21, 22, 23, 24, 25, 26, 27, 28, 29, or 30 nucleotides (nt) in each strand, wherein the double-stranded oligonucleotide comprises a double-stranded portion between 15 and 29 nucleotides long and either or both of the strands may comprise a 3′ overhang between, e.g., 1-5 nucleotides long, or either or both ends can be blunt. In some embodiments, an siRNA comprises strands between 19 and 25 nt, e.g., between 21 and 23 nucleotides long, wherein one or both strands comprises a 3′ overhang of 1-2 nucleotides. One strand of the double-stranded portion of the siRNA (termed the “guide strand” or “antisense strand”) is substantially complementary (e.g., at least 80% or more, e.g., 85%, 90%, 95%, or 100%) complementary to (e.g., having 3, 2, 1, or 0 mismatched nucleotide(s)) a target region in the mRNA, and the other double-stranded portion is substantially complementary to the first double-stranded portion. In many embodiments, the guide strand is 100% complementary to a target region in an mRNA and the other passenger strand is 100% complementary to the first double-stranded portion (it is understood that, in various embodiments, the 3′ overhang portion of the guide strand, if present, may or may not be complementary to the mRNA when the guide strand is hybridized to the mRNA). In some embodiments, a shRNA molecule is a nucleic acid molecule comprising a stem-loop, wherein the double-stranded stem is 16-30 nucleotides long and the loop is about 1-10 nucleotides long. siRNA can comprise a wide variety of modified nucleosides, nucleoside analogs and can comprise chemically or biologically modified bases, modified backbones, etc. Without limitation, any modification recognized in the art as being useful for RNAi can be used. Some modifications result in increased stability, cell uptake, potency, etc. Some modifications result in decreased immunogenicity or clearance. In certain embodiments the siRNA comprises a duplex about 19-23 (e.g., 19, 20, 21, 22, or 23) nucleotides in length and, optionally, one or two 3′ overhangs of 1-5 nucleotides in length, which may be composed of deoxyribonucleotides. shRNA comprise a single nucleic acid strand that contains two complementary portions separated by a predominantly non-selfcomplementary region. The complementary portions hybridize to form a duplex structure and the non-selfcomplementary region forms a loop connecting the 3′ end of one strand of the duplex and the 5′ end of the other strand. shRNAs undergo intracellular processing to generate siRNAs. Typically, the loop is between 1 and 8, e.g., 2-6 nucleotides long.

MicroRNAs (miRNAs) are small, naturally occurring, non-coding, single-stranded RNAs of about 21-25 nucleotides (in mammalian systems) that inhibit gene expression in a sequence-specific manner. They are generated intracellularly from precursors (pre-miRNA) having a characteristic secondary structure comprised of a short hairpin (about 70 nucleotides in length) containing a duplex that often includes one or more regions of imperfect complementarity which is in turn generated from a larger precursor (pri-miRNA). Naturally occurring miRNAs are typically only partially complementary to their target mRNA and often act via translational repression. RNAi agents modelled on endogenous miRNA or miRNA precursors are of use in certain embodiments of the invention. For example, an siRNA can be designed so that one strand hybridizes to a target mRNA with one or more mismatches or bulges mimicking the duplex formed by a miRNA and its target mRNA. Such siRNA may be referred to as miRNA mimics or miRNA-like molecules. miRNA mimics may be encoded by precursor nucleic acids whose structure mimics that of naturally occurring miRNA precursors.

In certain embodiments an RNAi agent is a vector (e.g., a plasmid or virus) that comprises a template for transcription of an siRNA (e.g., as two separate strands that can hybridize to each other), shRNA, or microRNA precursor. Typically the template encoding the siRNA, shRNA, or miRNA precursor is operably linked to expression control sequences (e.g., a promoter), as known in the art. Such vectors can be used to introduce the template into vertebrate cells, e.g., mammalian cells, and result in transient or stable expression of the siRNA, shRNA, or miRNA precursor. Precurors (shRNA or miRNA precursors) are processed intracellularly to generate siRNA or miRNA.

In general, small RNAi agents such as siRNA can be chemically synthesized or can be transcribed in vitro or in vivo from a DNA template either as two separate strands that then hybridize, or as an shRNA which is then processed to generate an siRNA. Often RNAi agents, especially those comprising modifications, are chemically synthesized. Chemical synthesis methods for oligonucleotides are well known in the art.

The term “small molecule” as used herein, is an organic molecule that is less than about 2 kilodaltons (KDa) in mass. In some embodiments, the small molecule is less than about 1.5 KDa, or less than about 1 KDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups. In some embodiments, a small molecule is non-polymeric. In some embodiments, a small molecule is not an amino acid. In some embodiments, a small molecule is not a nucleotide. In some embodiments, a small molecule is not a saccharide.

The term “subject” can be any multicellular animal. Often a subject is a vertebrate, e.g., a mammal or avian. Exemplary mammals include, e.g., humans, non-human primates, rodents (e.g., mouse, rat, rabbit), ungulates (e.g., ovine, bovine, equine, caprine species), canines, and felines. Often, a subject is an individual to whom a compound is to be delivered, e.g., for experimental, diagnostic, and/or therapeutic purposes or from whom a sample is obtained or on whom a diagnostic procedure is performed (e.g., a sample or procedure that will be used to assess tissue damage and/or to assess the effect of a compound of the invention).

The term “tissue damage” is used herein to refer to any type of damage or injury to cells, tissues, organs, or other body structures. The term encompasses, in various embodiments, degeneration due to disease, damage due to physical trauma or surgery, damage caused by exposure to deleterious substance, and other disruptions in the structure and/or functionality of cells, tissues, organs, or other body structures.

The term “tissue regeneration” or “TR” refers to at least partial regeneration, replacement, restoration, or regrowth of a tissue, organ, or other body structure, or portion thereof, following loss, damage, or degeneration, where said tissue regeneration but for the methods described in the present invention would not take place. Examples of tissue regeneration include the regrowth of severed digits or limbs including the regrowth of cartilage, bone, muscle, tendons, and ligaments, the scarless regrowth of bone, cartilage, skin, or muscle that has been lost due to injury or disease, with an increase in size and cell number of an injured or diseased organ such that the tissue or organ approximates the normal size of the tissue or organ or its size prior to injury or disease. Depending on the tissue type, tissue regeneration can occur via a variety of different mechanisms such as, for example, the rearrangement of pre-existing cells and/or tissue (e.g., through cell migration), the division of adult somatic stem cells or other progenitor cells and differentiation of at least some of their descendants, and/or the dedifferentiation, transdifferentiation, and/or proliferation of cells.

The term “partial reprogramming” is considered synonymous with iTR.

The term “TR activator genes” refers to genes whose lack of expression in fetal and adult cells but whose expression in embryonic phases of development facilitate TR.

The term “TR inhibitor genes” refers to genes whose expression in fetal and adult animals inhibit TR.

The term “treat”, “treating”, “therapy”, “therapeutic” and similar terms in regard to a subject refer to providing medical and/or surgical management of the subject. Treatment can include, but is not limited to, administering a compound or composition (e.g., a pharmaceutical composition) to a subject. Treatment of a subject according to the instant invention is typically undertaken in an effort to promote regeneration, e.g., in a subject who has suffered tissue damage or is expected to suffer tissue damage (e.g., a subject who will undergo surgery). The effect of treatment can generally include increased regeneration, reduced scarring, and/or improved structural or functional outcome following tissue damage (as compared with the outcome in the absence of treatment), and/or can include reversal or reduction in severity or progression of a degenerative disease.

The term “variant” as applied to a particular polypeptide refers to a polypeptide that differs from such polypeptide (sometimes referred to as the “original polypeptide”) by one or more amino acid alterations, e.g., addition(s), deletion(s), and/or substitution(s). Sometimes an original polypeptide is a naturally occurring polypeptide (e.g., from human or non-human animal) or a polypeptide identical thereto. Variants may be naturally occurring or created using, e.g., recombinant DNA techniques or chemical synthesis. An addition can be an insertion within the polypeptide or an addition at the N- or C-terminus. In some embodiments, the number of amino acids substituted, deleted, or added can be for example, about 1 to 30, e.g., about 1 to 20, e.g., about 1 to 10, e.g., about 1 to 5, e.g., 1, 2, 3, 4, or 5. In some embodiments, a variant comprises a polypeptide whose sequence is homologous to the sequence of the original polypeptide over at least 50 amino acids, at least 100 amino acids, at least 150 amino acids, or more, up to the full length of the original polypeptide (but is not identical in sequence to the original polypeptide), e.g., the sequence of the variant polypeptide is at least 50%, 60%, 70%, 75%, 80%, 85%, 90%, 95%, 96%, 97%, 98%, 99%, or more identical to the sequence of the original polypeptide over at least 50 amino acids, at least 100 amino acids, at least 150 amino acids, or more, up to the full length of the original polypeptide. In some embodiments, a variant comprises a polypeptide at least 50%, 60%, 70%, 75%, 80%, 85%, 90%, 91%, 92%, 93%, 94%, 95%, 96%, 97%, 98%, 99%, 99.5% or more identical to an original polypeptide over at least 50%, 60%, 70%, 80%, 85%, 90%, 91%, 92%, 93%, 94%, 95%, 96%, 97%, 98%, 99%, or 100% of the length of the original polypeptide. In some embodiments, a variant comprises at least one functional or structural domain, e.g., a domain identified as such in the Conserved Domain Database (CDD) of the National Center for Biotechnology Information (www.ncbi.nih.gov), e.g., an NCBI-curated domain.

In some embodiments one, more than one, or all biological functions or activities of a variant or fragment is substantially similar to that of the corresponding biological function or activity of the original molecule. In some embodiments, a functional variant retains at least 10%, 20%, 30%, 40%, 50%, 60%, 70%, 80%, 90%, 95%, 96%, 97%, 98%, 99%, or more of the activity of the original polypeptide, e.g., about equal activity. In some embodiments, the activity of a variant is up to approximately 100%, approximately 125%, or approximately 150% of the activity of the original molecule. In other nonlimiting embodiments, an activity of a variant or fragment is considered substantially similar to the activity of the original molecule if the amount or concentration of the variant needed to produce a particular effect is within 0.5 to 5-fold of the amount or concentration of the original molecule needed to produce that effect.

In some embodiments amino acid “substitutions” in a variant are the result of replacing one amino acid with another amino acid having similar structural and/or chemical properties, i.e., conservative amino acid replacements. “Conservative” amino acid substitutions may be made on the basis of similarity in any of a variety or properties such as side chain size, polarity, charge, solubility, hydrophobicity, hydrophilicity, and/or amphipathicity of the residues involved. For example, the non-polar (hydrophobic) amino acids include alanine, leucine, isoleucine, valine, glycine, proline, phenylalanine, tryptophan and methionine. The polar (hydrophilic), neutral amino acids include serine, threonine, cysteine, tyrosine, asparagine, and glutamine. The positively charged (basic) amino acids include arginine, lysine and histidine. The negatively charged (acidic) amino acids include aspartic acid and glutamic acid. Within a particular group, certain substitutions may be of particular interest, e.g., replacements of leucine by isoleucine (or vice versa), serine by threonine (or vice versa), or alanine by glycine (or vice versa). Of course, non-conservative substitutions are often compatible with retaining function as well. In some embodiments, a substitution or deletion does not alter or delete an amino acid important for activity. Insertions or deletions may range in size from about 1 to 20 amino acids, e.g., 1 to 10 amino acids. In some instances, larger domains may be removed without substantially affecting function. In certain embodiments of the invention the sequence of a variant can be obtained by making no more than a total of 5, 10, 15, or 20 amino acid additions, deletions, or substitutions to the sequence of a naturally occurring enzyme. In some embodiments no more than 1%, 5%, 10%, or 20% of the amino acids in a polypeptide are insertions, deletions, or substitutions relative to the original polypeptide. Guidance in determining which amino acid residues may be replaced, added, or deleted without eliminating or substantially reducing activities of interest, may be obtained by comparing the sequence of the particular polypeptide with that of homologous polypeptides (e.g., from other organisms) and minimizing the number of amino acid sequence changes made in regions of high homology (conserved regions) or by replacing amino acids with those found in homologous sequences since amino acid residues that are conserved among various species are more likely to be important for activity than amino acids that are not conserved.

In some embodiments, a variant of a polypeptide comprises a heterologous polypeptide portion. The heterologous portion often has a sequence that is not present in or homologous to the original polypeptide. A heterologous portion may be, e.g., between 5 and about 5,000 amino acids long, or longer. Often it is between 5 and about 1,000 amino acids long. In some embodiments, a heterologous portion comprises a sequence that is found in a different polypeptide, e.g., a functional domain. In some embodiments, a heterologous portion comprises a sequence useful for purifying, expressing, solubilizing, and/or detecting the polypeptide. In some embodiments, a heterologous portion comprises a polypeptide “tag”, e.g., an affinity tag or epitope tag. For example, the tag can be an affinity tag (e.g., HA, TAP, Myc, 6×His, Flag, GST), fluorescent or luminescent protein (e.g., EGFP, ECFP, EYFP, Cerulean, DsRed, mCherry), solubility-enhancing tag (e.g., a SUMO tag, NUS A tag, SNUT tag, or a monomeric mutant of the Ocr protein of bacteriophage T7). See, e.g., Esposito D and Chatterjee D K. Curr Opin Biotechnol.; 17(4):353-8 (2006). In some embodiments, a tag can serve multiple functions. A tag is often relatively small, e.g., ranging from a few amino acids up to about 100 amino acids long. In some embodiments a tag is more than 100 amino acids long, e.g., up to about 500 amino acids long, or more. In some embodiments, a polypeptide has a tag located at the N- or C-terminus, e.g., as an N- or C-terminal fusion. The polypeptide could comprise multiple tags. In some embodiments, a 6.times.His tag and a NUS tag are present, e.g., at the N-terminus. In some embodiments, a tag is cleavable, so that it can be removed from the polypeptide, e.g., by a protease. In some embodiments, this is achieved by including a sequence encoding a protease cleavage site between the sequence encoding the portion homologous to the original polypeptide and the tag. Exemplary proteases include, e.g., thrombin, TEV protease, Factor Xa, PreScission protease, etc. In some embodiments, a “self-cleaving” tag is used. See, e.g., PCT/US05/05763. Sequences encoding a tag can be located 5′ or 3′ with respect to a polynucleotide encoding the polypeptide (or both). In some embodiments, a tag or other heterologous sequence is separated from the rest of the polypeptide by a polypeptide linker. For example, a linker can be a short polypeptide (e.g., 15-25 amino acids). Often a linker is composed of small amino acid residues such as serine, glycine, and/or alanine. A heterologous domain could comprise a transmembrane domain, a secretion signal domain, etc.

In certain embodiments of the invention a fragment or variant, optionally excluding a heterologous portion, if present, possesses sufficient structural similarity to the original polypeptide so that when its 3-dimensional structure (either actual or predicted structure) is superimposed on the structure of the original polypeptide, the volume of overlap is at least 70%, preferably at least 80%, more preferably at least 90% of the total volume of the structure of the original polypeptide. A partial or complete 3-dimensional structure of the fragment or variant may be determined by crystallizing the protein, which can be done using standard methods. Alternately, an NMR solution structure can be generated, also using standard methods. A modeling program such as MODELER (Sali, A. and Blundell, T L, J. Mol. Biol., 234, 779-815, 1993), or any other modeling program, can be used to generate a predicted structure. If a structure or predicted structure of a related polypeptide is available, the model can be based on that structure. The PROSPECT-PSPP suite of programs can be used (Guo, J T, et al., Nucleic Acids Res. 32 (Web Server issue):W522-5, Jul. 1, 2004). Where embodiments of the invention relate to variants of a polypeptide, it will be understood that polynucleotides encoding the variant are provided.

The term “vector” is used herein to refer to a nucleic acid or a virus or portion thereof (e.g., a viral capsid or genome) capable of mediating entry of, e.g., transferring, transporting, etc., a nucleic acid molecule into a cell such as the host cell such as hES cells, hEC cells, or hES-derived embryonic progenitor cell lines producing the EVs where the vector is a nucleic acid, the nucleic acid molecule to be transferred is generally linked to, e.g., inserted into, the vector nucleic acid molecule. A nucleic acid vector may include sequences that direct autonomous replication (e.g., an origin of replication), or may include sequences sufficient to allow integration of part or all of the nucleic acid into host cell DNA. Useful nucleic acid vectors include, for example, DNA or RNA plasmids, cosmids, and naturally occurring or modified viral genomes or portions thereof or nucleic acids (DNA or RNA) that can be packaged into viral) capsids. Plasmid vectors typically include an origin of replication and one or more selectable markers. Plasmids may include part or all of a viral genome (e.g., a viral promoter, enhancer, processing or packaging signals, etc.). Viruses or portions thereof that can be used to introduce nucleic acid molecules into cells are referred to as viral vectors. Useful viral vectors include adenoviruses, adeno-associated viruses, retroviruses, lentiviruses, vaccinia virus and other poxviruses, herpesviruses (e.g., herpes simplex virus), and others. Viral vectors may or may not contain sufficient viral genetic information for production of infectious virus when introduced into host cells, i.e., viral vectors may be replication-defective, and such replication-defective viral vectors may be preferable for therapeutic use. Where sufficient information is lacking it may, but need not be, supplied by a host cell or by another vector introduced into the cell. The nucleic acid to be transferred may be incorporated into a naturally occurring or modified viral genome or a portion thereof or may be present within the virus or viral capsid as a separate nucleic acid molecule. It will be appreciated that certain plasmid vectors that include part or all of a viral genome, typically including viral genetic information sufficient to direct transcription of a nucleic acid that can be packaged into a viral capsid and/or sufficient to give rise to a nucleic acid that can be integrated into the host cell genome and/or to give rise to infectious virus, are also sometimes referred to in the art as viral vectors. Vectors may contain one or more nucleic acids encoding a marker suitable for use in the identifying and/or selecting cells that have or have not been transformed or transfected with the vector. Markers include, for example, proteins that increase or decrease either resistance or sensitivity to antibiotics (e.g., an antibiotic-resistance gene encoding a protein that confers resistance to an antibiotic such as puromycin, hygromycin or blasticidin) or other compounds, enzymes whose activities are detectable by assays known in the art (e.g., beta.-galactosidase or alkaline phosphatase), and proteins or RNAs that detectably affect the phenotype of transformed or transfected cells (e.g., fluorescent proteins). Expression vectors are vectors that include regulatory sequence(s), e.g., expression control sequences such as a promoter, sufficient to direct transcription of an operably linked nucleic acid. Regulatory sequences may also include enhancer sequences or upstream activator sequences. Vectors may optionally include 5′ leader or signal sequences. Vectors may optionally include cleavage and/or polyadenylations signals and/or a 3′ untranslated regions. Vectors often include one or more appropriately positioned sites for restriction enzymes, to facilitate introduction into the vector of the nucleic acid to be expressed. An expression vector comprises sufficient cis-acting elements for expression; other elements required or helpful for expression can be supplied by the host cell or in vitro expression system.

Various techniques may be employed for introducing nucleic acid molecules into vesicles. Such techniques include chemical-facilitated transfection using compounds such as calcium phosphate, cationic lipids, cationic polymers, liposome-mediated transfection, non-chemical methods such as electroporation, particle bombardment, or microinjection, and infection with a virus that contains the nucleic acid molecule of interest (sometimes termed “transduction”). Markers can be used for the identification and/or selection of vesicles that have taken up the vector and, typically, express the nucleic acid. Vesicles can be maintained in appropriate media purified such as with the use of filtration, centrifugation, or magnetic bead isolation steps as described herein to establish a clinical-grade formulation.

Reprogramming using reprogramming factors such as KLF4, OCT4, SOX2, and MYC can reset various hallmarks of aging such as epigenetic profile, telomere length, and mitochondrial fitness to an embryonic state (1). Thus, it provides a means to derive young tissues from older donor cells as a source of cells for tissue regeneration (2). Recently, a report of partial reprogramming using pulsed in vivo expression of KLF4, OCT4, SOX2, and MYC in a progeria model in transgenic mice was shown to increase regeneration in response to muscle and pancreatic injury without tumor formation (3). However, there are practical barriers to such transgenic modalities for regulating the expression of reprogramming factors in vivo, especially in the case of humans, to achieving partial reprogramming without the loss of cell identity, unregulated growth and/or tumor formation. Extracellular vesicles (EVs) from germ-line and pluripotent cells have been previously disclosed by us as a means of reprogramming human somatic cells to pluripotency (U.S. Patent Application Publication 2017/0152475, titled “Methods of Reprogramming Animal Somatic Cells,” incorporated in its entirety herein by reference). In addition, we previously disclosed the use of exosomes generated by embryonic progenitor cell lines (US Published Patent Application US2016/0108368 A1) that are useful for inducing angiogenic activity and for stabilizing blood vessels.

EVs from germ-line and pluripotent cells as well as EVs from embryonic progenitor cell lines are described here as a modulator of EFT and NT for induced tissue regeneration (iTR) without the expression of pluripotency factors from exogenous transgenes. EVs from germ-line or pluripotent cells include exosomes purified from EC cells. EC cells are a preferred embodiment as a source of pluripotent cell-derived EVs because of the ease of genetic modification, clonal expansion, scale up of the cells. In the case of producing iTR EVs, the EC cell line designated ReCytel is modified by the exogenous introduction of TERT, LIN28B SOX2, MYC, NANOG, and OCT4 such that the levels of the corresponding mRNAs are expressed at least 2-fold, or more preferably at least 10-fold, and most preferably, at least 50-fold higher than the native cell. Reduced immunogenicity of the EVs may also be accomplished by knockout of the Class I HLA antigens such as through the knockout of beta2-microglobulin and the overexpression of HLA-G. Exosomes are isolated from the cell supernatant of the resulting cells cultured in standard cell culture vessels or bioreactors, by differential centrifugation followed by ultracentrifugation. Additional isolation methods known in the art include the use of density gradient centrifugation, the use of a cushion, size exclusion filtration, or precipitation. A preferred purification method employs the use of immunomagnetic isolation techniques directed to exosomal antigens such as the tetraspanins C9 or CD81.

Exosomes from embryonic progenitor cells are used to reprogram cells to an “embryonic like” state (pre-EFT (embryonic to fetal transition)) as indicated by a decrease in the post-EFT marker COX7A1 (4).

In one aspect of the invention, COX7A1 gene expression is used as a marker of the embryonic to fetal transition (EFT).

COX7A1 is down-regulated very early in the process of reprogramming fibroblasts to pluripotent stem cells. Pluripotent stem cells (hES and iPS) as well as their differentiated progeny have little or no COX7A expression (4). COX7A1 levels remain minimal whether the progeny that have been partially differentiated as in embryonic progenitor cell lines (4) or fully differentiated to mature phenotypes such as adipocytes (4, 8). If low COX7A1 expression is an indicator of the embryonic state then reprogramming would be predicted to reset the higher COX7A1 levels observed in F/A cells to low levels observed in embryonic stem cells and in cells derived from embryonic stem cells (embryonic progenitors). Even partially reprogrammed cells would have low levels of COX7A1 if they need to pass through a partially differentiated (progenitor-like state) prior to becoming pluripotent.

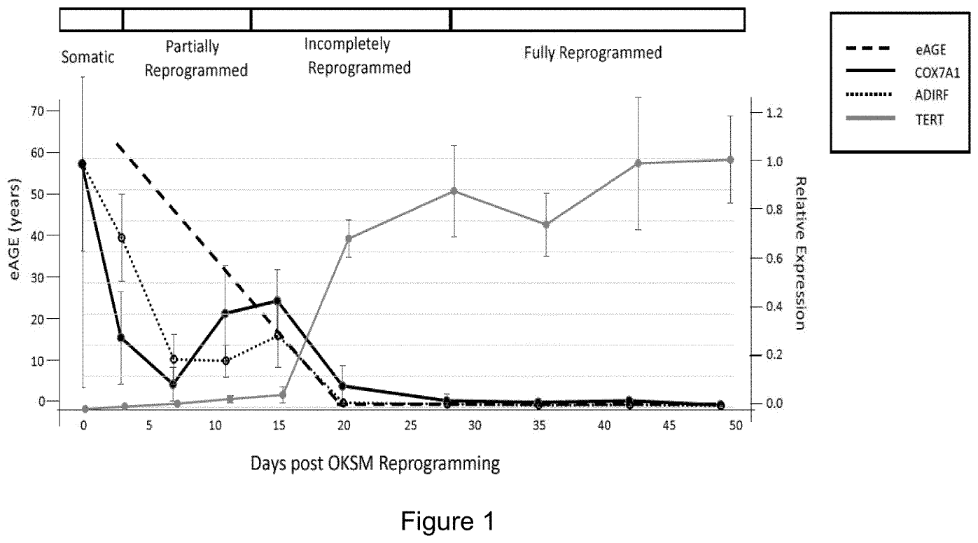

The change in COX7A1 expression was plotted over time after initiation of reprogramming using the whole transcriptome microarray expression data from Ohnuke (9). The change in COX7A1 expression was plotted with reprogramming stage and included the change in epigenetic age as plotted by Olova (10). As shown in FIG. 1 and Table 1, COX7A1 levels have declined to ˜10% of starting levels by day 7 when cells have reached a “partially reprogrammed state” as described by Tanabe where they have begun to express pluripotency related genes but are still capable of reverting to their initial cell identity (11). At day 7, the reprogrammed cells have decreased their epigenetic age by about 10 years (FIGS. 1 and (10)). The COX7A1 levels then increase at day 10 and 15 before returning to day 7 levels at day 20 (an incompletely reprogrammed state). Thus, a rapid decrease in COX7A1 gene expression by ˜90% within one week is useful for identifying cells that have been partially reprogrammed and rejuvenated to a younger epigenetic state. Partial reprogramming has been described as a more regenerative state and shown in-vivo to result in improved tissue regeneration and reduction in scar formation without tumor formation (12-17). The transient increase in COX7A1 expression at day 11 and 15 before cells are completely reprogrammed indicates that even moderate COX7A1 expression is incompatible with the reprogrammed state.

The extracellular vesicles of the present invention, while expected to be relatively non-immunogenic, may nevertheless have lower immune observability by reducing the levels of Class I HLA antigens including beta-2 microglobulin, and/or expressing HLA-G in the host cell International Patent Application Publication WO/2014/022423, titled “HLA G-Modified Cells and Methods,” which is incorporated herein in its entirety by reference.

Other inventive methods comprise use of EVs containing iTR factors in the ex vivo production of living, functional tissues, organs, or cell-containing compositions to repair or replace a tissue or organ lost due to damage. For example, cells or tissues removed from an individual (either the future recipient, an individual of the same species, or an individual of a different species) may be cultured in vitro, optionally with an matrix, scaffold (e.g., a three dimensional scaffold) or mold (e.g., comprising a biocompatible, optionally biodegradable, material, e.g., a polymer such as HyStem-C), and their development into a functional tissue or organ can be promoted by contacting EVs containing iTR factors. The scaffold, matrix, or mold may be composed at least in part of naturally occurring proteins such as collagen, hyaluronic acid, or alginate (or chemically modified derivatives of any of these), or synthetic polymers or copolymers of lactic acid, caprolactone, glycolic acid, etc., or self-assembling peptides, or decellularized matrices derived from tissues such as heart valves, intestinal mucosa, blood vessels, and trachea. In some embodiments, the scaffold comprises a hydrogel. The scaffold may, in certain embodiments, be coated or impregnated with vesicles containing iTR factors, which may diffuse out from the scaffold over time. After production ex vivo, the tissue or organ is grafted into or onto a subject. For example, the tissue or organ can be implanted or, in the case of certain tissues such as skin, placed on a body surface. The tissue or organ may continue to develop in vivo. In some embodiments, the tissue or organ to be produced at least in part ex vivo is a bladder, blood vessel, bone, fascia, liver, muscle, skin patch, etc. Suitable scaffolds may, for example, mimic the extracellular matrix (ECM).

Optionally, EVs containing iTR factors are administered to the subject prior to, during, and/or following grafting of the ex vivo generated tissue or organ. In some aspects, a biocompatible material is a material that is substantially non-toxic to cells in vitro at the concentration used or, in the case of a material that is administered to a living subject, is substantially nontoxic to the subject's cells in the quantities and at the location used and does not elicit or cause a significant deleterious or untoward effect on the subject, e.g., an immunological or inflammatory reaction, unacceptable scar tissue formation, etc. It will be understood that certain biocompatible materials may elicit such adverse reactions in a small percentage of subjects, typically less than about 5%, 1%, 0.5%, or 0.1%.

In some embodiments, a matrix or scaffold coated or impregnated with EVs such as exosomes loaded with iTR factors including those capable of causing a global pattern of iTR gene expression is implanted, optionally in combination with cells, into a subject in need of regeneration. The matrix or scaffold may be in the shape of a tissue or organ whose regeneration is desired. The cells may be stem cells of one or more type(s) that gives rise to such tissue or organ and/or of type(s) found in such tissue or organ.

In some embodiments, EVs containing iTR factors are administered directly to or near a site of tissue damage. “Directly to a site of tissue damage” encompasses injecting a compound or composition into a site of tissue damage or spreading, pouring, or otherwise directly contacting the site of tissue damage with the compound or composition. In some embodiments, administration is considered “near a site of tissue damage” if administration occurs within up to about 10 cm away from a visible or otherwise evident edge of a site of tissue damage or to a blood vessel (e.g., an artery) that is located at least in part within the damaged tissue or organ. Administration “near a site of tissue damage” is sometimes administration within a damaged organ, but at a location where damage is not evident. In some embodiments, following damage or loss of a tissue, organ, or other structure, EVs containing iTR factors is applied to the remaining portion of the tissue, organ, or other structure. In some embodiments, EVs containing iTR factors are applied to the end of a severed digit or limb) that remains attached to the body, to enhance regeneration of the portion that has been lost. In some embodiments, the severed portion is reattached surgically, and EVs containing iTR factors are applied to either or both faces of the wound. In some embodiments, EVs containing iTR factors are administered to enhance engraftment or healing or regeneration of a transplanted organ or portion thereof. In some embodiments, EVs containing iTR factors are used to enhance nerve regeneration. For example, EVs containing iTR factors may be infused into a severed nerve, e.g., near the proximal and/or distal stump. In some embodiments, EVs containing iTR factors are placed within an artificial nerve conduit, a tube composed of biological or synthetic materials within which the nerve ends and intervening gap are enclosed. The EVs containing iTR factors may be formulated in a matrix to facilitate their controlled release over time. Said matrix may comprise a biocompatible, optionally biodegradable, material, e.g., a polymer such as that comprised of hyaluronic acid, including crosslinked hyaluronic acid or carboxymethyl hyaluronate crosslinked with PEGDA, or a mixture of carboxymethyl hyaluronate crosslinked by PEGDA with carboxymethyl-modified gelatin (HyStem-C).

In some embodiments, the EVs containing iTR factors contain both mRNAs for TERT as well as LIN28B, OCT4, SOX2, MYC, and NANOG described herein and may or may not be formulated for localization and slow release in carboxymethyl hyaluronate crosslinked by PEGDA with carboxymethyl-modified gelatin (HyStem-C) to induce iTR.

iTM and iCM factors such as mRNA to COX7A1 can similarly be loaded into exosomes derived from fetal or adult cells can be administered in physiological solutions such as saline, or slow-released in carboxymethyl hyaluronate crosslinked by PEGDA with carboxymethyl-modified gelatin (HyStem-C) to induce iTM or iCM.

In some embodiments, EVs containing iTR factors or combinations of factors is used to promote production of hair follicles and/or growth of hair. In some embodiments, EVs containing iTR factors triggers regeneration of hair follicles from epithelial cells that do not normally form hair. In some embodiments, EVs containing iTR factors are used to treat hair loss, hair sparseness, partial or complete baldness in a male or female. In some embodiments, baldness is the state of having no or essentially no hair or lacking hair where it often grows, such as on the top, back, and/or sides of the head. In some embodiments, hair sparseness is the state of having less hair than normal or average or, in some embodiments, less hair than an individual had in the past or, in some embodiments, less hair than an individual considers desirable. In some embodiments, EVs containing iTR factors are used to promote growth of eyebrows or eyelashes. In some embodiments, EVs containing iTR factors are used to treat androgenic alopecia or “male pattern baldness” (which can affect males and females). In some embodiments, EVs containing iTR factors are used to treat alopecia areata, which involves patchy hair loss on the scalp, alopecia totalis, which involves the loss of all head hair, or alopecia universalis, which involves the loss of all hair from the head and the body. In some embodiments, EVs containing iTR factors are applied to a site where hair growth is desired, e.g., the scalp or eyebrow region. In some embodiments, EVs containing iTR factors are applied to or near the edge of the eyelid, to promote eyelash growth. In some embodiments, EVs containing iTR factors are applied in a liquid formulation. In some embodiments, EVs containing iTR factors are applied in a cream, ointment, paste, or gel. In some embodiments, EVs containing iTR factors are used to enhance hair growth after a burn, surgery, chemotherapy, or other event causing loss of hair or hear-bearing skin.

In some embodiments, EVs containing iTR factors are administered to tissues afflicted with age-related degenerative changes to regenerate youthful function. Said age-related degenerative changes includes by way of nonlimiting example, age-related macular degeneration, coronary disease, osteoporosis, osteonecrosis, heart failure, emphysema, peripheral artery disease, vocal cord atrophy, hearing loss, Alzheimer's disease, Parkinson's disease, skin ulcers, and other age-related degenerative diseases. In some embodiments, said EVs containing iTR factors include mRNA encoding the catalytic component of telomerase to extend cell lifespan.

In some embodiments, EVs containing iTR factors are administered to enhance replacement of cells that have been lost or damaged due to insults such as chemotherapy, radiation, or toxins. In some embodiments, such cells are stromal cells of solid organs and tissues.

Inventive methods of treatment can include a step of identifying or providing a subject suffering from or at risk of a disease or condition in which in which enhancing regeneration would be of benefit to the subject. In some embodiments, the subject has experienced injury (e.g., physical trauma) or damage to a tissue or organ. In some embodiments, the damage is to a limb or digit. In some embodiments, a subject suffers from a disease affecting the cardiovascular, digestive, endocrine, musculoskeletal, gastrointestinal, hepatic, integumentary, nervous, respiratory, or urinary system. In some embodiments, tissue damage is to a tissue, organ, or structure such as cartilage, bone, heart, blood vessel, esophagus, stomach, liver, gallbladder, pancreas, intestines, rectum, anus, endocrine gland, skin, hair follicle, tooth, gum, lip, nose, mouth, thymus, spleen, skeletal muscle, smooth muscle, joint, brain, spinal cord, peripheral nerve, ovary, fallopian tube, uterus, vagina, mammary gland, testes, vas deferens, seminal vesicle, prostate, penis, pharynx, larynx, trachea, bronchi, lungs, kidney, ureter, bladder, urethra, eye (e.g., retina, cornea), or ear (e.g., organ of Corti).

In some embodiments EVs containing iTR factors are administered to a subject at least once within approximately 2, 4, 8, 12, 24, 48, 72, or 96 hours after a subject has suffered tissue damage (e.g., an injury or an acute disease-related event such as a myocardial infarction or stroke) and, optionally, at least once thereafter. In some embodiments, EVs containing iTR factors are administered to a subject at least once within approximately 1-2 weeks, 2-6 weeks, or 6-12 weeks, after a subject has suffered tissue damage and, optionally, at least once thereafter.

In some embodiments of the invention, it may useful to stimulate or facilitate regeneration or de novo development of a missing or hypoplastic tissue, organ, or structure by, for example, removing the skin, removing at least some tissue at a site where regeneration or de novo development is desired, abrading a joint or bone surface where regeneration or de novo development is desired, and/or inflicting another type of wound on a subject. In the case of regeneration after tissue damage, it may be desirable to remove (e.g., by surgical excision or debridement) at least some of the damaged tissue. In some embodiments, EVs containing iTR factors are administered at or near the site of such removal or abrasion.

In some embodiments, EVs containing iTR factors are used to enhance generation of a tissue or organ in a subject in whom such tissue or organ is at least partially absent as a result of a congenital disorder, e.g., a genetic disease. Many congenital malformations result in hypoplasia or absence of a variety of tissues, organs, or body structures such as limbs or digits. In other instances, a developmental disorder resulting in hypoplasia of a tissue, organ, or other body structure becomes evident after birth. In some embodiments, EVs containing iTR factors are administered to a subject suffering from hypoplasia or absence of a tissue, organ, or other body structure, in order to stimulate growth or development of such tissue, organ, or other body structure. In some aspects, the invention provides a method of enhancing generation of a tissue, organ, or other body structure in a subject suffering from hypoplasia or congenital absence of such tissue, organ, or other body structure, the method comprising administering EVs containing iTR factors to the subject. In some embodiments, EVs containing iTR factors are administered to the subject prior to birth, i.e., in utero. The various aspects and embodiments of the invention described herein with respect to regeneration are applicable to such de novo generation of a tissue, organ, or other body structure and are encompassed within the invention.

In some aspects, EVs containing iTR factors are used to enhance generation of tissue in any of a variety of situations in which new tissue growth is useful at locations where such tissue did not previously exist. For example, generating bone tissue between joints is frequently useful in the context of fusion of spinal or other joints.

EVs containing iTR factors may be tested in a variety of animal models of regeneration. In one aspect, EVs containing iTR factors are tested in murine species. For example, mice can be wounded (e.g., by incision, amputation, transection, or removal of a tissue fragment). EVs containing iTR factors are applied to the site of the wound and/or to a removed tissue fragment and its effect on regeneration is assessed. The effect of a modulator of vertebrate TR can be tested in a variety of vertebrate models for tissue or organ regeneration. For example, fin regeneration can be assessed in zebrafish, e.g., as described in (Mathew L K, Unraveling tissue regeneration pathways using chemical genetics. J Biol Chem. 282(48):35202-10 (2007)), and can serve as a model for limb regeneration. Rodent, canine, equine, caprine, fish, amphibian, and other animal models useful for testing the effects of treatment on regeneration of tissues and organs such as heart, lung, limbs, skeletal muscle, bone, etc., are widely available. For example, various animal models for musculoskeletal regeneration are discussed in Tissue Eng Part B Rev. 16(1) (2010). A commonly used animal model for the study of liver regeneration involves surgical removal of a larger portion of the rodent liver. Other models for liver regeneration include acute or chronic liver injury or liver failure caused by toxins such as carbon tetrachloride. In some embodiments, a model for hair regeneration or healing of skin wounds involves excising a patch of skin, e.g., from a mouse. Regeneration of hair follicles, hair growth, re-epithelialization, gland formation, etc., can be assessed.

The compounds and compositions disclosed herein and/or identified using a method and/or assay system described herein may be administered by any suitable means such as orally, intranasally, subcutaneously, intramuscularly, intravenously, intra-arterially, parenterally, intraperitoneally, intrathecally, intratracheally, ocularly, sublingually, vaginally, rectally, dermally, or by inhalation, e.g., as an aerosol. The particular mode selected will depend, of course, upon the particular compound selected, the particular condition being treated and the dosage required for therapeutic efficacy. The methods of this invention, generally speaking, may be practiced using any mode of administration that is medically or veterinarily acceptable, meaning any mode that produces acceptable levels of efficacy without causing clinically unacceptable (e.g., medically or veterinarily unacceptable) adverse effects. Suitable preparations, e.g., substantially pure preparations, of one or more compound(s) may be combined with one or more pharmaceutically acceptable carriers or excipients, etc., to produce an appropriate pharmaceutical composition suitable for administration to a subject. Such pharmaceutically acceptable compositions are an aspect of the invention. The term “pharmaceutically acceptable carrier or excipient” refers to a carrier (which term encompasses carriers, media, diluents, solvents, vehicles, etc.) or excipient which does not significantly interfere with the biological activity or effectiveness of the active ingredient(s) of a composition and which is not excessively toxic to the host at the concentrations at which it is used or administered. Other pharmaceutically acceptable ingredients can be present in the composition as well. Suitable substances and their use for the formulation of pharmaceutically active compounds are well-known in the art (see, for example, “Remington's Pharmaceutical Sciences”, E. W. Martin, 19th Ed., 1995, Mack Publishing Co.: Easton, Pa., and more recent editions or versions thereof, such as Remington: The Science and Practice of Pharmacy. 21st Edition. Philadelphia, Pa. Lippincott Williams & Wilkins, 2005, for additional discussion of pharmaceutically acceptable substances and methods of preparing pharmaceutical compositions of various types). Furthermore, compounds and compositions of the invention may be used in combination with any compound or composition used in the art for treatment of a particular disease or condition of interest.

A pharmaceutical composition is typically formulated to be compatible with its intended route of administration. For example, preparations for parenteral administration include sterile aqueous or non-aqueous solutions, suspensions, and emulsions. Aqueous carriers include water, alcoholic/aqueous solutions, emulsions or suspensions, including saline and buffered media, e.g., sodium chloride solution, Ringer's dextrose, dextrose and sodium chloride, lactated Ringer's. Examples of non-aqueous solvents are propylene glycol, polyethylene glycol, vegetable oils such as olive oil, and injectable organic esters such as ethyl oleate. fixed oils, polyethylene glycols, glycerine, propylene glycol or other synthetic solvents; preservatives, e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. pH can be adjusted with acids or bases, such as hydrochloric acid or sodium hydroxide. Such parenteral preparations can be enclosed in ampoules, disposable syringes or multiple dose vials made of glass or plastic.

For administration by inhalation, inventive compositions may be delivered in the form of an aerosol spray from a pressured container or dispenser that contains a suitable propellant, e.g., a gas such as carbon dioxide, a fluorocarbon, or a nebulizer. Liquid or dry aerosol (e.g., dry powders, large porous particles, etc.) can be used. The present invention also contemplates delivery of compositions using a nasal spray or other forms of nasal administration.

For topical applications, pharmaceutical compositions may be formulated in a suitable ointment, lotion, gel, or cream containing the active components suspended or dissolved in one or more pharmaceutically acceptable carriers suitable for use in such composition.

For local delivery to the eye, the pharmaceutically acceptable compositions may be formulated as solutions or micronized suspensions in isotonic, pH adjusted sterile saline, e.g., for use in eye drops, or in an ointment, or for intra-ocularly administration, e.g., by injection.

Pharmaceutical compositions may be formulated for transmucosal or transdermal delivery. For transmucosal or transdermal administration, penetrants appropriate to the barrier to be permeated may be used in the formulation. Such penetrants are generally known in the art. Inventive pharmaceutical compositions may be formulated as suppositories (e.g., with conventional suppository bases such as cocoa butter and other glycerides) or as retention enemas for rectal delivery.

In some embodiments, a composition includes one or more agents intended to protect the active agent(s) against rapid elimination from the body, such as a controlled release formulation, implants, microencapsulated delivery system, etc. Compositions may incorporate agents to improve stability (e.g., in the gastrointestinal tract or bloodstream) and/or to enhance absorption. Compounds may be encapsulated or incorporated into particles, e.g., microparticles or nanoparticles. Biodegradable, biocompatible polymers can be used, such as ethylene vinyl acetate, polyanhydrides, polyglycolic acid, PLGA, collagen, polyorthoesters, polyethers, and polylactic acid. Methods for preparation of such formulations will be apparent to those skilled in the art. For example, and without limitation, a number of particle, lipid, and/or polymer-based delivery systems are known in the art for delivery of siRNA. The invention contemplates use of such compositions. Liposomes or other lipid-based particles can also be used as pharmaceutically acceptable carriers.

Pharmaceutical compositions and compounds for use in such compositions may be manufactured under conditions that meet standards, criteria, or guidelines prescribed by a regulatory agency. For example, such compositions and compounds may be manufactured according to Good Manufacturing Practices (GMP) and/or subjected to quality control procedures appropriate for pharmaceutical agents to be administered to humans and can be provided with a label approved by a government regulatory agency responsible for regulating pharmaceutical, surgical, or other therapeutically useful products.

Pharmaceutical compositions of the invention, when administered to a subject for treatment purposes, are preferably administered for a time and in an amount sufficient to treat the disease or condition for which they are administered. Therapeutic efficacy and toxicity of active agents can be assessed by standard pharmaceutical procedures in cell cultures or experimental animals. The data obtained from cell culture assays and animal studies can be used in formulating a range of dosages suitable for use in humans or other subjects. Different doses for human administration can be further tested in clinical trials in humans as known in the art. The dose used may be the maximum tolerated dose or a lower dose. A therapeutically effective dose of an active agent in a pharmaceutical composition may be within a range of about 0.001 mg/kg to about 100 mg/kg body weight, about 0.01 to about 25 mg/kg body weight, about 0.1 to about 20 mg/kg body weight, about 1 to about 10 mg/kg. Other exemplary doses include, for example, about 1 μg/kg to about 500 mg/kg, about 100 μg/kg to about 5 mg/kg. In some embodiments, a single dose is administered while in other embodiments multiple doses are administered. Those of ordinary skill in the art will appreciate that appropriate doses in any particular circumstance depend upon the potency of the agent(s) utilized, and may optionally be tailored to the particular recipient. The specific dose level for a subject may depend upon a variety of factors including the activity of the specific agent(s) employed, the particular disease or condition and its severity, the age, body weight, general health of the subject, etc. It may be desirable to formulate pharmaceutical compositions, particularly those for oral or parenteral compositions, in unit dosage form for ease of administration and uniformity of dosage. Unit dosage form, as that term is used herein, refers to physically discrete units suited as unitary dosages for the subject to be treated; each unit containing a predetermined quantity of active agent(s) calculated to produce the desired therapeutic effect in association with an appropriate pharmaceutically acceptable carrier. It will be understood that a therapeutic regimen may include administration of multiple doses, e.g., unit dosage forms, over a period of time, which can extend over days, weeks, months, or years. A subject may receive one or more doses a day, or may receive doses every other day or less frequently, within a treatment period. For example, administration may be biweekly, weekly, etc. Administration may continue, for example, until appropriate structure and/or function of a tissue or organ has been at least partially restored and/or until continued administration of the compound does not appear to promote further regeneration or improvement. In some embodiments, a subject administers one or more doses of a composition of the invention to him or herself.

In some embodiments, two or more compounds or compositions are administered in combination, e.g., for purposes of enhancing regeneration. Compounds or compositions administered in combination may be administered together in the same composition, or separately. In some embodiments, administration “in combination” means, with respect to administration of first and second compounds or compositions, administration performed such that (i) a dose of the second compound is administered before more than 90% of the most recently administered dose of the first agent has been metabolized to an inactive form or excreted from the body; or (ii) doses of the first and second compound are administered within 48, 72, 96, 120, or 168 hours of each other, or (iii) the agents are administered during overlapping time periods (e.g., by continuous or intermittent infusion); or (iv) any combination of the foregoing. In some embodiments, two or more EVs containing iTR factors expressing the catalytic component of telomerase and an iTR factor, are administered. In some embodiments EVs containing iTR factors are administered in combination with a combination with one or more growth factors, growth factor receptor ligands (e.g., agonists), hormones (e.g., steroid or peptide hormones), or signaling molecules, useful to promote regeneration and polarity. Of particular utility are organizing center molecules useful in organizing regeneration competent cells such as those produced using the methods of the present invention. In some embodiments, a growth factor is an epidermal growth factor family member (e.g., EGF, a neuregulin), a fibroblast growth factor (e.g., any of FGF1-FGF23), a hepatocyte growth factor (HGF), a nerve growth factor, a bone morphogenetic protein (e.g., any of BMP1-BMP7), a vascular endothelial growth factor (VEGF), a wnt ligand, a wnt antagonist, retinoic acid, NOTUM, follistatin, sonic hedgehog, or other organizing center factors.

Veterinary Applications

The compositions and methods of the present invention are applicable to homologous application in veterinary medicine wherein the host cells producing the EVs and mRNAs are from the corresponding species.

Targeting and Fusogenic Modifications

The host cells producing the EVs may also be genetically modified to express targeting and fusogenic that promote the specific targeting of EVs to a desired target cell type as well as proteins that promote the fusion of the EVs with the target cell plasma membrane. Examples of cell type specific targeting proteins are the clustered protocadherin genes that provide a cell-type specific adhesion. Additional targeting peptides and proteins include growth factors such as FGFs and EGF and antibodies with affinity for cell-type specific antigens that bind and internalize are used for targeting EVs carrying iTR factors to specific cells, tissues or organs. Targeting peptides, antibodies and antibody fragments selected from large combinatorial libraries of peptides displayed on a genetic package that links the gene encoding the targeting peptide/protein with the peptide/protein itself (phage, ribosome and yeast display) are well known in the art. For example, the heart targeting peptide CRPPR is used as a targeting peptide to enrich accumulation of therapeutics to the heart (Zhang et al Biomaterials (2008) 29(12): 1976-88). Examples of fusogenic proteins include HPV16 E5 protein.

Stabilization of mRNAs

The mRNAs that are loaded into isolated EVs such as exosomes of the present invention may be modified such as by the replacement of uridine by pseudouridine to increase their stability, decrease innate immune responses, and increase functional half-life.

### Example 1: Conditioned Medium from Embryonic Progenitor Endothelial Cells Down Regulates COX7A1 in Fetal/Adult Human Vascular Endothelial Cells

Secreted factors in the conditioned medium from clonal embryonic endothelial progenitor cell eEPC lines were tested to determine if they could confer an embryonic gene expression pattern onto fetal/adult endothelial cells. Medium (EGM-MV2 basal medium supplemented with VEGF, IGF2, and FGF2 (VIF)) from 3 clonal eEPC cell lines (30-MV2-10, 30-MV2-17, and 30-MV2-19) was collected after a 72-hour incubation with cells at 37° C., 5% 02, 10% CO2. HUVEC (human umbilical cord vein cells) or HBMVEC (human brain microvascular endothelial cells) at ˜80% confluency were washed 2× with PBS and their growth medium was replaced with conditioned medium from 30-MV2-10, 30-MV2-17, or 30-MV2-19. Cells were incubated for 72 hours in conditioned medium before harvesting for RNA extraction. RNA was isolated and gene expression assessed by quantitative rtPCR using standard methods.

The change in COX7A1 gene expression relative to its expression in cells incubated in growth medium was calculated using the AACT method. Statistical significance was determined using a one-way ANOVA test. Incubation of HUVEC cells in eEPC conditioned medium resulted in a significant reduction of COX7A1 gene expression compared to normal growth medium or exosome free medium (basal medium with VIF and 5% exosome free calf serum). Incubation of HBMVEC cells with eEPC conditioned medium also resulted in a significant reduction in COX7A1 expression with 30-MV2-19 conditioned medium resulting in the greatest reduction of COX7A1 expression. These results indicate that the secretome of embryonic progenitor cells contains factors capable of conferring a more embryonic phenotype onto fetal adult cells as indicated by the EFT marker, COX7A1.

### Example 2: Exosomes from Embryonic Progenitor Cells Down Regulate COX7A1 in Adult Endothelial Cells

Exosome enriched fractions from the conditioned medium of embryonic endothelial progenitor cell lines (30-MV2-10, 30-MV2-14 and 30-MV2-19) were prepared by ultrafiltration of the conditioned medium that was prepared as described in Example 1. The medium was centrifuged at 300×g for 5 min. at room temperature followed by 1000 xg for 10 min. followed by filtration through a 0.22 uM filter. The filtered medium was centrifuged at 10,000×g for 10 minutes at 4° C. The medium was then concentrated 300× by centrifugation through an Amicon Ultra-70 100 kDa cutoff device at 1000 xg for 1 hour. The HUVEC were plated at ˜80% confluency and incubated in EGM-MV2 medium containing exosome free FCS.

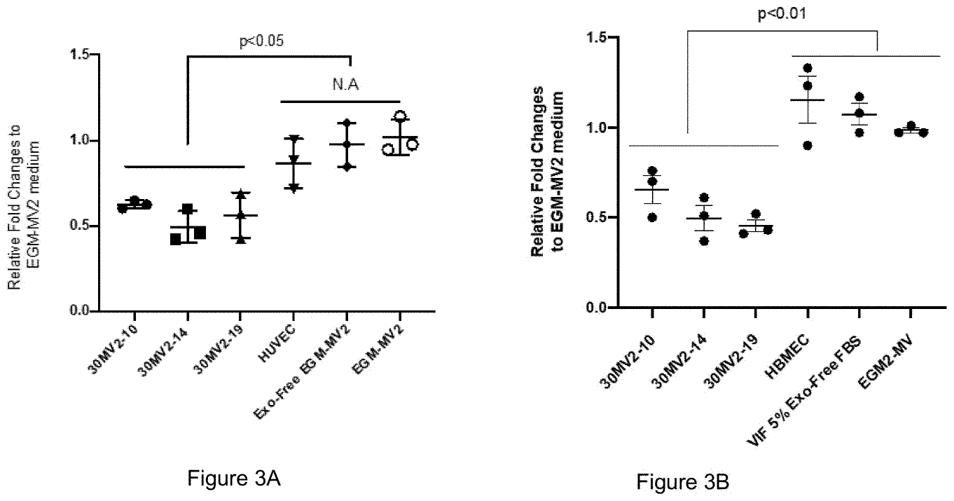

The exosome enriched preparations from embryonic or HUVEC cell conditioned media were added to a final concentration of 10× (1:30 dilution) at day 0 and at day 3. The conditioning medium (exosome free medium) without exposure to cells and exosomes concentrated from HUVEC conditioned medium were used as controls. The cells were harvested on day 6 for RNA preparation. Gene expression assessed by quantitative rtPCR using standard methods and change in COX7A1 was calculated using the AACT method. As shown in FIG. 3, incubation with exosome enriched medium from 3 embryonic endothelial progenitor cells resulted in an up to 50% decrease in COX7A1 expression.

TABLE 1
Decrease in COX7A1 Expression During Reprogramming

| Reprogramming | Epigenetic | COX7A1 | |
|---|---|---|---|
| Time | Age | Expression | |
| (days) | (years) | (% of Max) | Cell State |
| 0 | 60 | 100 | Somatic |
| 3 | 57 | 30 | Transitional |
| 7 | 50 | 11 | Partially Reprogrammed |
| 11 | 34 | 40 | Transitional |
| 15 | 17 | 45 | Incompletely |
| | | | Reprogrammed |
| 20 | 0 | 10 | Incompletely |
| | | | Reprogrammed |
| 28 | 0 | 4 | Transitional |
| 35-49 | 0 | 4 | Fully Reprogrammed |

### Example 3. Use of Genetically-Modified Human Embryonal Carcinoma Cells as a Source of Exosomes for Use in ITR

EC cells are a preferred embodiment as a source of pluripotent cell-derived EVs because of the ease of genetic modification, clonal expansion, scale up of the cells. In the case of producing iTR EVs from EC cells, the EC cell line designated ReCytel is modified by the exogenous introduction of combinations of TERT, LIN28B SOX2, MYC, NANOG, and OCT4 such that the levels of the corresponding mRNAs are expressed at least 2-fold, or more preferably at least 10-fold, and most preferably, at least 50-fold higher than the native cell. Reduced immunogenicity of the EVs is also be accomplished by knockout of the Class I HLA antigen function through the knockout of beta2-microglobulin and the overexpression of HLA-G. Exosomes are isolated from the cell supernatant of the resulting cells cultured in standard cell culture vessels or bioreactors, by differential centrifugation followed by ultracentrifugation and the use of immunomagnetic isolation techniques directed to exosomal antigens such as the tetraspanins C9 or CD81. The resulting exosomes are useful in inducing tissue regeneration in humans and for veterinary use in inducing regeneration in tissues otherwise not capable of regeneration.

### Example 4. Use of Native Human Embryonal Carcinoma Cells as a Source of Exosomes for Use in ITR

EC cells are a preferred embodiment as a source of pluripotent cell-derived EVs because of the ease of scale up of the cells. In the case of producing iTR EVs from EC cells, the EC cell line designated ReCytel is modified to reduce immunogenicity of the EVs and is accomplished by knockout of the Class I HLA antigen function through the knockout of beta2-microglobulin and the overexpression of HLA-G. Exosomes are isolated from the cell supernatant of the resulting cells cultured in standard cell culture vessels or bioreactors, by differential centrifugation followed by ultracentrifugation and the use of immunomagnetic isolation techniques directed to exosomal antigens such as the tetraspanins C9 or CD81. The resulting exosomes are then loaded with exogenous mRNA for combinations of TERT, LIN28B SOX2, MYC, NANOG, and OCT4 such that the levels of the corresponding mRNAs are expressed at least 2-fold, or more preferably at least 10-fold, and most preferably, at least 50-fold higher than the native cell. The resulting loaded exosomes are useful in inducing tissue regeneration in humans and for veterinary use in inducing regeneration in tissues otherwise not capable of regeneration.

Embodiments of the invention also include:

1. A method for inducing cell or tissue regeneration, comprised of the steps: 1) isolating extracellular vesicles from host cells that display a pre-fetal pattern of gene expression; 2) contacting cells and/or tissue in vivo with the extracellular vesicles to restore a pre-fetal pattern of gene expression and induce tissue regeneration, but without reprogramming said cells and tissues to pluripotency.

2. The method of claim 1, wherein the more embryonic phenotype is indicated by a decrease in expression of COX7A1 from the reprogrammed cells and/or tissue.

3. The method of claim 1, wherein the cells are initially adult human cells.

4. The method of claim 1, wherein the contacted cells and/or tissue have a COX7A expression of less than 10% maximum by at least day 20.

5. The method of claim 1, wherein the extracellular vesicles are exosomes.

6. The method of claim 1, wherein the extracellular vesicles are derived from human embryonal carcinoma cells or embryonic progenitor cells.

7. A method for inducing cell or tissue regeneration, comprised of the steps: 1) isolating extracellular vesicles from host embryonal carcinoma cells that are genetically-modified to overexpress LIN28B, SOX2, NANOG, OCT4 and MYC; 2) contacting cells and/or tissue in vivo with the extracellular vesicles to restore a pre-fetal pattern of gene expression and induce tissue regeneration, but without reprogramming said cells and tissues to pluripotency.

8. The method of claim 7, wherein the more embryonic phenotype is indicated by a decrease in expression of COX7A1 from the reprogrammed cells and/or tissue.

9. The method of claim 7, wherein the cells are initially adult cells.

10. The method of claim 7, wherein the contacted cells and/or tissue have a COX7A1 expression of less than 10% maximum by at least day 20.

11. The method of claim 7, wherein the extracellular vesicles are exosomes.

12. A method for inducing cell or tissue regeneration, comprised of the steps: 1) isolating extracellular vesicles from host cells that display a pre-fetal pattern of gene expression; 2) introducing into said extracellular vesicles mRNAs for LIN28B, SOX2, NANOG, OCT4 and MYC; 3) contacting cells and/or tissue in vivo with the extracellular vesicles to restore a pre-fetal pattern of gene expression and induce tissue regeneration, but without reprogramming said cells and tissues to pluripotency.

13. The method of claim 12, wherein the more embryonic phenotype is indicated by a decrease in expression of COX7A1 from the reprogrammed cells and/or tissue.

14. The method of claim 12, wherein the cells are initially adult cells.

15. The method of claim 12, wherein the contacted cells and/or tissue have a COX7A1 expression of less than 10% maximum by at least day 20.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

In the context of the present invention, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order that is logically possible.

### REFERENCES

1. Mahmoudi, S., and Brunet, A. (2012) Aging and reprogramming: a two-way street. Curr Opin Cell Biol 24, 744-756
2. Lapasset, L., Milhavet, O., Prieur, A., Besnard, E., Babied, A., Ait-Hamou, N., Leschik, J., Pellestor, F., Ramirez, J. M., De Vos, J., Lehmann, S., and Lemaitre, J. M. (2011) Rejuvenating senescent and centenarian human cells by reprogramming through the pluripotent state. Genes Dev 25, 2248-2253
3. Ocampo, A., Reddy, P., Martinez-Redondo, P., Platero-Luengo, A., Hatanaka, F., Hishida, T., Li, M., Lam, D., Kurita, M., Beyret, E., Araoka, T., Vazquez-Ferrer, E., Donoso, D., Roman, J. L., Xu, J., Rodriguez Esteban, C., Nunez, G., Nunez Delicado, E., Campistol, J. M., Guillen, I., Guillen, P., and Izpisua Belmonte, J. C. (2016) In Vivo Amelioration of Age-Associated Hallmarks by Partial Reprogramming. Cell 167, 1719-1733 e1712
4. West, M. D., Labat, I., Sternberg, H., Larocca, D., Nasonkin, I., Chapman, K., Singh, R., Larocca, J., Makarev, E., Aliper, A. M., Kazennov, A., Alekseenko, A., Shuvalov, N., Cheskidova, E., Alekseev, A., Artemov, A., Putin, E., Mamoshina, P., Nikita P., Copeland, K., Korzinkin, M., Lezhnina, K., Izumchenko, E., Zhavoronkov, A. (2017) Use of Deep Neural Network Ensembles to Identify Embryonic-Fetal Transition Markers: Repression of COX7A1 in Embryonic and Cancer Cells. Oncotarget
5. Zhou, W., Choi, M., Margineantu, D., Margaretha, L., Hesson, J., Cavanaugh, C., Blau, C. A., Horwitz, M. S., Hockenbery, D., Ware, C., and Ruohola-Baker, H. (2012) HIFlalpha induced switch from bivalent to exclusively glycolytic metabolism during ESC-to-EpiSC/hESC transition. EMBO J 31, 2103-2116
6. Mishra, N., Timilsina, U., Ghimire, D., Dubey, R. C., and Gaur, R. (2017) Downregulation of cytochrome c oxidase subunit 7A1 expression is important in enhancing cell proliferation in adenocarcinoma cells. Biocher Biophys Res Commun 482, 713-719
7. West, M. D., Sargent, R. G., Long, J., Brown, C., Chu, J. S., Kessler, S., Derugin, N., Sampathkumar, J., Burrows, C., Vaziri, H., Williams, R., Chapman, K. B., Larocca, D., Loring, J. F., and Murai, J. (2008) The ACTCellerate initiative: large-scale combinatorial cloning of novel human embryonic stem cell derivatives. Regen Med 3, 287-308
8. West, M. D., Chang, C. F., Larocca. D., Li, J., Jiang, J., Sim, P., Labat, I., Chapman, K. B., Wong, K. E., Nicoll, J., Van Kanegan, M. J., de Grey, A., Nasonkin, I. O., Stahl, A., and Sternberg, H. (2019) Clonal derivation of white and brown adipocyte progenitor cell lines from human pluripotent stem cells. Stem Cell Res Ther 10, 7
9. Ohnuki, M., Tanabe, K., Sutou, K., Teramoto, I., Sawamura, Y., Narita, M., Nakamura, M., Tokunaga, Y., Nakamura, M., Watanabe, A., Yamanaka, S., and Takahashi, K. (2014) Dynamic regulation of human endogenous retroviruses mediates factor-induced reprogramming and differentiation potential. Proc Natl Acad Sci USA 111, 12426-12431
10. Olova, N., Simpson, D. J., Marioni, R. E., and Chandra, T. (2019) Partial reprogramming induces a steady decline in epigenetic age before loss of somatic identity. Aging Cell 18, e12877
11. Tanabe, K., Nakamura, M., Narita, M., Takahashi, K., and Yamanaka, S. (2013) Maturation, not initiation, is the major roadblock during reprogramming toward pluripotency from human fibroblasts. Proc Natl Acad Sci USA 110, 12172-12179
12. Kurita, M., Araoka, T., Hishida, T., O'Keefe, D. D., Takahashi, Y., Sakamoto, A., Sakurai, M., Suzuki, K., Wu, J., Yamamoto, M., Hernandez-Benitez, R., Ocampo, A., Reddy, P., Shokhirev, M. N., Magistretti, P., Nunez Delicado, E., Eto, H., Harii, K., and Izpisua Belmonte, J. C. (2018) In vivo reprogramming of wound-resident cells generates skin epithelial tissue. Nature 561, 243-247
13. Tang, Y., and Cheng, L. (2017) Cocktail of chemical compounds robustly promoting cell reprogramming protects liver against acute injury. Protein Cell 8, 273-283
14. Marion, R. M., and Blasco, M. A. (2017) Long Live Partial Reprogramming. Circ Res 120, 1381-1383
15. Tamanini, S., Comi, G. P., and Corti, S. (2018) In Vivo Transient and Partial Cell Reprogramming to Pluripotency as a Therapeutic Tool for Neurodegenerative Diseases. Mol Neurobiol 55, 6850-6862
16. Beyret, E., Martinez Redondo, P., Platero Luengo, A., and Izpisua Belmonte, J. C. (2018) Elixir of Life: Thwarting Aging With Regenerative Reprogramming. Circ Res 122, 128-141
17. Doeser, M. C., Scholer, H. R., and Wu, G. (2018) Reduction of Fibrosis and Scar Formation by Partial Reprogramming In Vivo. Stem Cells 36, 1216-1225

1. A method for inducing cell or tissue regeneration, comprised of the steps: 1) isolating extracellular vesicles from host cells that display a pre-fetal pattern of gene expression; 2) contacting cells and/or tissue in vivo with the extracellular vesicles to restore a pre-fetal pattern of gene expression and induce tissue regeneration, but without reprogramming said cells and tissues to pluripotency.

2. The method of claim 1, wherein the more embryonic phenotype is indicated by a decrease in expression of COX7A1 from the reprogrammed cells and/or tissue.

3. The method of claim 1, wherein the cells are initially adult human cells.

4. The method of claim 1, wherein the contacted cells and/or tissue have a COX7A1 expression of less than 10% maximum by at least day 20.

5. The method of claim 1, wherein the extracellular vesicles are exosomes.

6. The method of claim 1, wherein the extracellular vesicles are derived from human embryonal carcinoma cells or embryonic progenitor cells.

7. A method for inducing cell or tissue regeneration, comprised of the steps: 1) isolating extracellular vesicles from host embryonal carcinoma cells that are genetically-modified to overexpress LIN28B, SOX2, NANOG, OCT4 and MYC; 2) contacting cells and/or tissue in vivo with the extracellular vesicles to restore a pre-fetal pattern of gene expression and induce tissue regeneration, but without reprogramming said cells and tissues to pluripotency.

8. The method of claim 7, wherein the more embryonic phenotype is indicated by a decrease in expression of COX7A1 from the reprogrammed cells and/or tissue.

9. The method of claim 7, wherein the cells are initially adult cells.

10. The method of claim 7, wherein the contacted cells and/or tissue have a COX7A1 expression of less than 10% maximum by at least day 20.

11. The method of claim 7, wherein the extracellular vesicles are exosomes.

12. A method for inducing cell or tissue regeneration, comprised of the steps: 1) isolating extracellular vesicles from host cells that display a pre-fetal pattern of gene expression; 2) introducing into said extracellular vesicles mRNAs for LIN28B, SOX2, NANOG, OCT4 and MYC; 3) contacting cells and/or tissue in vivo with the extracellular vesicles to restore a pre-fetal pattern of gene expression and induce tissue regeneration, but without reprogramming said cells and tissues to pluripotency.

13. The method of claim 12, wherein the more embryonic phenotype is indicated by a decrease in expression of COX7A1 from the reprogrammed cells and/or tissue.

14. The method of claim 12, wherein the cells are initially adult cells.

15. The method of claim 12, wherein the contacted cells and/or tissue have a COX7A1 expression of less than 10% maximum by at least day 20.
